(19) United States
(12) Patent Application Publication
(10) Pub. No.: US 2022/0017621 A1
(43) Pub. Date: Jan. 20, 2022

(54) Combination Therapy for Cancer

(71) Applicant: Merck Patent GmbH, Darmstadt (DE)

(72) Inventors: Kin-Ming Lo, Lexington, MA (US); Yan Lan, Belmont, MA (US)

(21) Appl. No.: 17/357,380

(22) Filed: Jun. 24, 2021

Related U.S. Application Data
(63) Continuation of application No. 15/674,655, filed on Aug. 11, 2017, now abandoned.
(60) Provisional application No. 62/374,621, filed on Aug. 12, 2016.

(51) Int. Cl. C07K 16/28; A61K 31/555; A61K 45/06; C07K 14/705; A61K 38/17; A61K 39/395; A61P 35/02; A61P 35/00; A61K 31/282; A61K 31/513; A61N 5/10; C07K 14/71; C07K 16/30

(52) U.S. Cl. CPC C07K 16/2827; A61K 2039/505; A61K 45/06; C07K 14/70596; A61K 38/179; A61K 39/39558; A61P 35/02; A61P 35/00; A61K 31/282; A61K 31/513; A61K 39/3955; A61N 5/1077; C07K 14/71; C07K 16/30; C07K 2319/00; C07K 2317/24; C07K 2317/31; C07K 2317/76; A61K 31/555

(57) ABSTRACT

This invention relates generally to a combination therapy for the treatment of cancer, particularly to a combination of (i) a bifunctional molecule comprising a TGFβRII or fragment thereof capable of binding TGFβ and an antibody, or antigen binding fragment thereof, that binds to an immune checkpoint protein, such as Programmed Death Ligand 1 (PD-L1) and (ii) at least one additional anti-cancer therapeutic agent.

### CROSS-REFERENCE TO RELATED APPLICATIONS

This application is a continuation of U.S. patent application Ser. No. 15/674,655, filed Aug. 11, 2017, which claims priority to and the benefit of U.S. Provisional Patent Application No. 62/374,621 filed Aug. 12, 2016, the entire contents of each of which are incorporated by reference herein.

### FIELD OF THE INVENTION

This invention relates generally to a combination therapy for the treatment of cancer, particularly to a combination of (i) a bifunctional molecule comprising a TGFβRII or fragment thereof capable of binding TGFβ and an antibody, or antigen binding fragment thereof, that binds to an immune checkpoint protein, such as Programmed Death Ligand 1 (PD-L1) and (ii) at least one additional anti-cancer therapeutic agent. Anti-cancer therapeutic agents include, for example, radiation, chemotherapeutic agents, biologics, or vaccines. In certain embodiments of the invention, the combination therapy provides for a synergistic anti-cancer effect.

### BACKGROUND

In cancer treatment, it has long been recognized that chemotherapy is associated with high toxicity and can lead to emergence of resistant cancer cell variants. Most chemotherapeutic agents cause undesirable side effects including cardiac and renal toxicity, alopecia, nausea and vomiting. Radiation therapy is also used in cancer treatment. Such treatment uses high-energy particles or waves, such as x-rays, gamma rays, electron beams, or protons, to destroy or damage cancer cells. Unlike chemotherapy, which exposes the whole body to cancer-fighting drugs, radiation therapy is more commonly a local treatment. However, it is difficult to selectively administer therapeutic radiation only to the abnormal tissue and, thus, normal tissue near the abnormal tissue is also exposed to potentially damaging doses of radiation throughout treatment.

Cancer immunotherapy is a new paradigm in cancer treatment that instead of targeting cancer cells focuses on the activation of the immune system. Its principle is to rearm the host's immune response, especially the adaptive T cell response, to provide immune surveillance to kill the cancer cells, in particular, the minimal residual disease that has escaped other forms of treatment, hence achieving long-lasting protective immunity.

FDA approval of the anti-CTLA-4 antibody ipilimumab for the treatment of melanoma in 2011 ushered in a new era of cancer immunotherapy. The demonstration that anti-PD-1 or anti-PD-L1 therapy induced durable responses in melanoma, kidney, and lung cancer in clinical trials further signify its coming of age (Pardoll, D. M., Nat Immunol. 2012; 13:1129-32). However, ipilimumab therapy is limited by its toxicity profile, presumably because anti-CTLA-4 treatment, by interfering with the primary T cell inhibitory checkpoint, can lead to the generation of new autoreactive T cells. While inhibiting the PD-L1/PD-1 interaction results in dis-inhibiting existing chronic immune responses in exhausted T cells that are mostly antiviral or anticancer in nature (Wherry, E. J., Nat Immunol. 2011; 12:492-9), anti-PD-1 therapy can nevertheless sometimes result in potentially fatal lung-related autoimmune adverse events. Despite the promising clinical activities of anti-PD1 and anti-PD-L1 so far, increasing the therapeutic index, either by increasing therapeutic activity or decreasing toxicity, or both, remains a central goal in the development of immunotherapeutics.

### SUMMARY OF THE INVENTION

The present invention is based on the discovery of a combination therapy for cancer that includes administration of a bifunctional protein containing at least a portion of TGFβ Receptor II (TGFβRII) that is capable of binding TGFβ and an antibody, or antigen-binding fragment, that binds to an immune checkpoint protein such as human protein Programmed Death Ligand 1 (PD-L1). The combination therapy also includes administration of an anti-cancer therapeutic agent such as, for example, radiation, chemotherapeutic agents, a biologic and/or a vaccine. The combination therapy exhibits a synergistic effect as compared to the effect of administering the individual agents separately.

Accordingly, in a first aspect, the present invention features a method of treating cancer in a subject that includes (i) administration of a bifunctional protein comprising a human TGFβRII, or a fragment thereof capable of binding TGFβ (e.g., a soluble fragment), and an antibody, or an antigen-binding fragment thereof, that binds PD-L1 (e.g., any of the antibodies or antibody fragments described herein); and (ii) administration of at least one additional second anti-cancer therapeutic agent.

In certain embodiments, the combination treatment method of the invention features the use of a polypeptide including (a) at least a variable domain of a heavy chain of an antibody that binds PD-L1 (e.g., amino acids 1-120 of SEQ ID NO: 2); and (b) human TGFβRII, or a soluble fragment thereof capable of binding TGFβ (e.g., a human TGFβRII extra-cellular domain (ECD), amino acids 24-159 of SEQ ID NO: 9, or any of those described herein) in combination with at least one additional anti-cancer therapeutic agent. The polypeptide may further include an amino acid linker connecting the C-terminus of the variable domain to the N-terminus of the human TGFβRII or soluble fragment thereof capable of binding TGFβ. The polypeptide may include the amino acid sequence of SEQ ID NO: 3 or an amino acid sequence substantially identical to SEQ ID NO: 3. The antibody fragment may be an scFv, Fab, F(ab′)₂, or Fv fragment.

In certain embodiments, the protein or polypeptide includes an antibody or antigen-binding fragment thereof that includes SEQ ID NO: 2 and human TGFβRII. The antibody may optionally include a modified constant region (e.g., any described herein, including a C-terminal Lys→Ala substitution, a mutation of the Leu-Ser-Leu-Ser (SEQ ID NO: 19) sequence to Ala-Thr-Ala-Thr (SEQ ID NO: 20), or a hybrid constant region including an IgG1 hinge region and an IgG2 CH2 domain).

In certain embodiments, the protein or polypeptide includes an antibody or antigen-binding fragment thereof that includes SEQ ID NO: 2 and a fragment of human TGFβRII capable of binding TGFβ (e.g., a soluble fragment). The antibody may optionally include a modified constant region (e.g., any described herein, including a C-terminal Lys→Ala substitution, a mutation of the Leu-Ser-Leu-Ser (SEQ ID NO: 19) sequence to Ala-Thr-Ala-Thr (SEQ ID NO: 20), or a hybrid constant region including an IgG1 hinge region and an IgG2 CH2 domain).

In certain embodiments, the protein or polypeptide includes an antibody or antigen-binding fragment thereof that includes SEQ ID NO: 2 and a human TGFβRII ECD. The antibody may include a modified constant region (e.g., any described herein, including a C-terminal Lys→Ala substitution, a mutation of the Leu-Ser-Leu-Ser (SEQ ID NO: 19) sequence to Ala-Thr-Ala-Thr (SEQ ID NO: 20), or a hybrid constant region including an IgG1 hinge region and an IgG2 CH2 domain).

In certain embodiments, the protein or polypeptide includes an antibody or antigen-binding fragment thereof that includes amino acids 1-120 of SEQ ID NO: 2 and human TGFβRII. The antibody may include a modified constant region (e.g., any described herein, including a C-terminal Lys→Ala substitution, a mutation of the Leu-Ser-Leu-Ser (SEQ ID NO: 19) sequence to Ala-Thr-Ala-Thr (SEQ ID NO: 20), or a hybrid constant region including an IgG1 hinge region and an IgG2 CH2 domain).

In certain embodiments, the protein or polypeptide includes an antibody or antigen-binding fragment thereof that includes amino acids 1-120 of SEQ ID NO: 2 and a fragment of human TGFβRII capable of binding TGFβ (e.g., a soluble fragment). The antibody may include a modified constant region (e.g., any described herein, including a C-terminal Lys→Ala substitution, a mutation of the Leu-Ser-Leu-Ser (SEQ ID NO: 19) sequence to Ala-Thr-Ala-Thr (SEQ ID NO: 20), or a hybrid constant region including an IgG1 hinge region and an IgG2 CH2 domain).

In certain embodiments, the protein or polypeptide includes an antibody or antigen-binding fragment thereof that includes amino acids 1-120 of SEQ ID NO: 2 and a human TGFβRII ECD. The antibody may include a modified constant region (e.g., any described herein, including a C-terminal Lys→Ala substitution, a mutation of the Leu-Ser-Leu-Ser (SEQ ID NO: 19) sequence to Ala-Thr-Ala-Thr (SEQ ID NO: 20), or a hybrid constant region including an IgG1 hinge region and an IgG2 CH2 domain).

In certain embodiments, the protein or polypeptide includes an antibody or antigen-binding fragment thereof that includes the hypervariable regions present in SEQ ID NO: 2 and human TGFβRII. The antibody may include a modified constant region (e.g., any described herein, including a C-terminal Lys→Ala substitution, a mutation of the Leu-Ser-Leu-Ser (SEQ ID NO: 19) sequence to Ala-Thr-Ala-Thr (SEQ ID NO: 20), or a hybrid constant region including an IgG1 hinge region and an IgG2 CH2 domain).

In certain embodiments, the protein or polypeptide includes an antibody or antigen-binding fragment thereof that includes the hypervariable regions present in SEQ ID NO: 2 and a fragment of human TGFβRII capable of binding TGFβ (e.g., a soluble fragment). The antibody may include a modified constant region (e.g., any described herein, including a C-terminal Lys→Ala substitution, a mutation of the Leu-Ser-Leu-Ser (SEQ ID NO: 19) sequence to Ala-Thr-Ala-Thr (SEQ ID NO: 20), or a hybrid constant region including an IgG1 hinge region and an IgG2 CH2 domain).

In certain embodiments, the protein or polypeptide includes an antibody or antigen-binding fragment thereof that includes the hypervariable regions present in SEQ ID NO: 2 and a human TGFβRII ECD. The antibody may include a modified constant region (e.g., any described herein, including a C-terminal Lys→Ala substitution, a mutation of the Leu-Ser-Leu-Ser (SEQ ID NO: 19) sequence to Ala-Thr-Ala-Thr (SEQ ID NO: 20), or a hybrid constant region including an IgG1 hinge region and an IgG2 CH2 domain).

In certain embodiments, the protein or polypeptide includes an antibody or antigen-binding fragment thereof that includes SEQ ID NO: 12 and human TGFβRII. The antibody may include a modified constant region (e.g., any described herein, including a C-terminal Lys→Ala substitution, a mutation of the Leu-Ser-Leu-Ser (SEQ ID NO: 19) sequence to Ala-Thr-Ala-Thr (SEQ ID NO: 20), or a hybrid constant region including an IgG1 hinge region and an IgG2 CH2 domain).

In certain embodiments, the protein or polypeptide includes an antibody or antigen-binding fragment thereof that includes SEQ ID NO: 12 and a fragment of human TGFβRII capable of binding TGFβ (e.g., a soluble fragment). The antibody may include a modified constant region (e.g., any described herein, including a C-terminal Lys→Ala substitution, a mutation of the Leu-Ser-Leu-Ser (SEQ ID NO: 19) sequence to Ala-Thr-Ala-Thr (SEQ ID NO: 20), or a hybrid constant region including an IgG1 hinge region and an IgG2 CH2 domain).

In certain embodiments, the protein or polypeptide includes an antibody or antigen-binding fragment thereof that includes SEQ ID NO: 12 and a human TGFβRII ECD. The antibody may include a modified constant region (e.g., any described herein, including a C-terminal Lys→Ala substitution, a mutation of the Leu-Ser-Leu-Ser (SEQ ID NO: 19) sequence to Ala-Thr-Ala-Thr (SEQ ID NO: 20), or a hybrid constant region including an IgG1 hinge region and an IgG2 CH2 domain).

In certain embodiments, the protein or polypeptide includes an antibody or antigen-binding fragment thereof that includes the hypervariable regions present in SEQ ID NO: 12 and human TGFβRII. The antibody may include a modified constant region (e.g., any described herein, including a C-terminal Lys→Ala substitution, a mutation of the Leu-Ser-Leu-Ser (SEQ ID NO: 19) sequence to Ala-Thr-Ala-Thr (SEQ ID NO: 20), or a hybrid constant region including an IgG1 hinge region and an IgG2 CH2 domain).

In certain embodiments, the protein or polypeptide includes an antibody or antigen-binding fragment thereof that includes the hypervariable regions present in SEQ ID NO: 12 and a fragment of human TGFβRII capable of binding TGFβ (e.g., a soluble fragment). The antibody may include a modified constant region (e.g., any described herein, including a C-terminal Lys→Ala substitution, a mutation of the Leu-Ser-Leu-Ser (SEQ ID NO: 19) sequence to Ala-Thr-Ala-Thr (SEQ ID NO: 20), or a hybrid constant region including an IgG1 hinge region and an IgG2 CH2 domain).

In certain embodiments, the protein or polypeptide includes an antibody or antigen-binding fragment thereof that includes the hypervariable regions present in SEQ ID NO: 12 and a human TGFβRII ECD. The antibody may include a modified constant region (e.g., any described herein, including a C-terminal Lys→Ala substitution, a mutation of the Leu-Ser-Leu-Ser (SEQ ID NO: 19) sequence to Ala-Thr-Ala-Thr (SEQ ID NO: 20), or a hybrid constant region including an IgG1 hinge region and an IgG2 CH2 domain).

In certain embodiments, the protein or polypeptide includes an antibody or antigen-binding fragment thereof that includes SEQ ID NO: 14 and human TGFβRII. The antibody may include a modified constant region (e.g., any described herein, including a C-terminal Lys→Ala substitution, a mutation of the Leu-Ser-Leu-Ser (SEQ ID NO: 19) sequence to Ala-Thr-Ala-Thr (SEQ ID NO: 20), or a hybrid constant region including an IgG1 hinge region and an IgG2 CH2 domain).

In certain embodiments, the protein or polypeptide includes an antibody or antigen-binding fragment thereof that includes SEQ ID NO: 14 and a fragment of human TGFβRII capable of binding TGFβ (e.g., a soluble fragment). The antibody may include a modified constant region (e.g., any described herein, including a C-terminal Lys→Ala substitution, a mutation of the Leu-Ser-Leu-Ser (SEQ ID NO: 19) sequence to Ala-Thr-Ala-Thr (SEQ ID NO: 20), or a hybrid constant region including an IgG1 hinge region and an IgG2 CH2 domain).

In certain embodiments, the protein or polypeptide includes an antibody or antigen-binding fragment thereof that includes SEQ ID NO: 14 and a human TGFβRII ECD. The antibody may include a modified constant region (e.g., any described herein, including a C-terminal Lys→Ala substitution, a mutation of the Leu-Ser-Leu-Ser (SEQ ID NO: 19) sequence to Ala-Thr-Ala-Thr (SEQ ID NO: 20), or a hybrid constant region including an IgG1 hinge region and an IgG2 CH2 domain).

The invention also provides for the use, in the combination therapy of the invention, of a protein including the polypeptide described above and at least a variable domain of a light chain of an antibody which, when combined with the polypeptide, forms an antigen-binding site that binds PD-L1. The protein may include (a) two polypeptides, each having an amino acid sequence consisting of the amino acid sequence of SEQ ID NO: 3, and (b) two additional polypeptides each having an amino acid sequence consisting of the amino acid sequence of SEQ ID NO: 1.

The invention features a combination therapy for treatment of cancer which comprises the administration of a protein described above, in combination with administration of one or more additional anti-cancer therapeutic agents for use in treating cancer or for use in inhibiting tumor growth. The one or more additional anti-cancer therapeutic agents include radiation, a chemotherapeutic agent, a biologic, and/or a vaccine.

The cancer or tumor may be selected from the group consisting of colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, non-small cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodysplastic syndromes.

The invention also features a combination therapy method of inhibiting tumor growth or treating cancer. The method includes exposing the tumor to a protein described above. The method further includes exposing the tumor to radiation and or administration of a chemotherapeutic, a biologic, or a vaccine. In certain embodiments, the tumor or cancer is selected from the group consisting of colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, non-small cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodysplastic syndromes.

By “TGFβRII” or “TGFβ Receptor II” is meant a polypeptide having the wild-type human TGFβ Receptor Type 2 Isoform A sequence (e.g., the amino acid sequence of NCBI Reference Sequence (RefSeq) Accession No. NP_001020018 (SEQ ID NO: 8)), or a polypeptide having the wild-type human TGFβ Receptor Type 2 Isoform B sequence (e.g., the amino acid sequence of NCBI RefSeq Accession No. NP_003233 (SEQ ID NO: 9)) or having a sequence substantially identical the amino acid sequence of SEQ ID NO: 8 or of SEQ ID NO: 9. The TGFβRII may retain at least 0.1%, 0.5%, 1%, 5%, 10%, 25%, 35%, 50%, 75%, 90%, 95%, or 99% of the TGFβ-binding activity of the wild-type sequence. The polypeptide of expressed TGFβRII lacks the signal sequence.

By a “fragment of TGFβRII capable of binding TGFβ” is meant any portion of NCBI RefSeq Accession No. NP_001020018 (SEQ ID NO: 8) or of NCBI RefSeq Accession No. NP_003233 (SEQ ID NO: 9), or a sequence substantially identical to SEQ ID NO: 8 or SEQ ID NO: 9 that is at least 20 (e.g., at least 30, 40, 50, 60, 70, 80, 90, 100, 110, 120, 130, 140, 150, 160, 175, or 200) amino acids in length that retains at least some of the TGFβ-binding activity (e.g., at least 0.1%, 0.5%, 1%, 5%, 10%, 25%, 35%, 50%, 75%, 90%, 95%, or 99%) of the wild-type receptor or of the corresponding wild-type fragment. Typically such fragment is a soluble fragment. An exemplary such fragment is a TGFβRII extra-cellular domain having the sequence of SEQ ID NO: 10.

By “substantially identical” is meant a polypeptide exhibiting at least 50%, desirably 60%, 70%, 75%, or 80%, more desirably 85%, 90%, or 95%, and most desirably 99% amino acid sequence identity to a reference amino acid sequence. The length of comparison sequences will generally be at least 10 amino acids, desirably at least 15 contiguous amino acids, more desirably at least 20, 25, 50, 75, 90, 100, 150, 200, 250, 300, or 350 contiguous amino acids, and most desirably the full-length amino acid sequence.

By “patient” is meant either a human or non-human animal (e.g., a mammal).

By “treating” a disease, disorder, or condition (e.g., a cancer) in a patient is meant reducing at least one symptom of the disease, disorder, or condition by administrating a therapeutic agent to the patient.

By “cancer” is meant a collection of cells multiplying in an abnormal manner.

Other embodiments and details of the invention are presented herein below.

### BRIEF DESCRIPTION OF THE DRAWINGS

FIG. 1 is a schematic drawing of an anti-PD-L1/TGFβ Trap molecule comprising one anti-PD-L1 antibody fused to two extracellular domain (ECD) of TGFβ Receptor II via a (Gly₄Ser)₄Gly linker (SEQ ID NO:11).

FIG. 2 is a table summarizing the study design for study “TI13-027: Combination of Anti-PD-L1/TGFβ Trap with 5-FU and Oxaliplatin Therapy in MC38 Tumor Model in C57B/L6 Wild Type Mice” where group and treatment is N=10 mice/group.

FIG. 3 is a table summarizing the study design for study “TI14-012: Combination of Anti-PD-L1/TGFβ Trap with 5-FU and Oxaliplatin Therapy in MC38 Tumor Model in B Cell Deficient Mice” where group and treatment is N=10 mice/group.

FIG. 4A-4D are a series of graphs showing that the Oxaliplatin/5-FU and anti-PD-L1/TGFβ trap combination enhances tumor growth inhibition and tumor-reactive CD8⁺ T cell responses (C57BL/6 Mice; Study TI13-027). FIG. 4A and FIG. 4D Tumor volumes were measured twice per week throughout the study period. Tumor volume data was log transformed and a two-way, repeated measure ANOVA was performed. FIG. 4B. Tumor weight data was evaluated with one-way ANOVA. FIG. 4C. The frequency of IFN-γ producing, P15E-specific CD8⁺ T cells was quantified by ELISpot assay. ELISpot data was evaluated by one-way ANOVA. All ANOVA included Tukey's correction for multiple comparisons to measure statistical differences between treatment groups. p<0.05 was determined to be statistically significant.

FIG. 5A-5D are a series of graphs showing that the Oxaliplatin/5-FU and anti-PD-L1/TGFβ trap combination enhances tumor growth inhibition and tumor-reactive CD8⁺ T cell responses (B6.12952-Ighm^{tm1Cgn}/J Mice; Study TI14-012). FIGS. 5A and 4D. Tumor volume data was log transformed and a two-way, repeated measure ANOVA was performed. FIG. 5B. Tumor weight data was evaluated with one-way ANOVA. FIG. 5C. The frequency of IFN-γ producing, P15E-specific CD8⁺ T cells was quantified by ELISpot assay. ELISpot data was evaluated by one-way ANOVA. All ANOVA included Tukey's correction for multiple comparisons to measure statistical differences between treatment groups. p<0.05 was determined to be statistically significant.

FIG. 6A-6C are a series of graphs showing that radiation and anti-PD-L1/TGFβ trap induces synergistic tumor growth inhibition and tumor-reactive CD8⁺ T Cell responses (TI13-109). FIG. 6A. Tumor volumes were measured twice per week and the average tumor volumes were presented as the mean±standard error of the mean (SEM). FIG. 6B. Tumor weight data was determined on day 14. FIG. 6C. The frequency of IFN-γ producing, P15E-specific CD8⁺ T cells was quantified by ELISpot assay on day 14. The data of anti-PD-L1/TGFβ Trap at the dose level of 164 μg were similar to the data at the dose level of 55 μg, either as a monotherapy or the combination.

FIG. 7A-7C are a series of graphs showing that radiation and anti-PD-L1/TGFβ trap induces synergistic tumor growth inhibition and tumor-Reactive CD8⁺ T cell responses (repeat study) (TI14-013). FIG. 7A. Tumor volumes were measured twice per week and the average tumor volumes were presented as the mean±standard error of the mean (SEM). FIG. 7B. Tumor weights were evaluated on day 14. FIG. 7C. The frequency of IFN-γ producing, P15E-specific CD8⁺ T cells was quantified by ELISpot assay on day 14.

FIG. 8A-8D are a series of graphs showing that radiation and anti-PD-L1/TGFβ trap promotes tumor-infiltrating CD8⁺ T cells and NK cells (TI14-013). FIG. 8A. Tumor-infiltrating CD8⁺ TILS. FIG. 8B. Tumor-infiltrating NK1.1⁺ TILS. FIG. 8C. CD8⁺ TIL EOMES Expression. FIG. 8D. CD8⁺ TIL Degranulation.

FIG. 9A is a schematic diagram demonstrating the administration of radiation in a mouse carrying a primary and secondary tumor in order to test for an abscopal effect.

FIG. 9B is a line graph showing primary tumor volume in mice in the days since the start of treatment.

FIG. 9C is a line graph showing secondary tumor volume (mm³) in mice in the days since the start of treatment. (●=Isotype control 400 μg; ♦=Anti-PDL1-TGFβ Trap μg; ▪=Radiation 500 rads; ▾=Radiation+Anti-PDL1-TGFβ Trap).

### DETAILED DESCRIPTION

This invention relates generally to a combination therapy for the treatment of cancer, particularly to a combination of (i) a bifunctional molecule comprising a TGFβRII or fragment thereof capable of binding TGFβ and an antibody, or antigen binding fragment thereof, that binds to an immune checkpoint protein, such as Programmed Death Ligand 1 (PD-L1) and (ii) at least one additional anti-cancer therapeutic agent. Such anti-cancer therapeutic agents include, for example, radiation, chemotherapeutic agents, a biologic, and/or a vaccine. In certain embodiments of the invention, the combination therapy provides for a synergistic anti-cancer effect.

The combination therapy of the invention is particularly advantageous, since not only the anti-cancer effect is enhanced compared to the effect of each agent alone, but the dosage of the one or more agents in a combination therapy can be reduced as compared to monotherapy with each agent, while still achieving an overall anti-cancer effect. Due to the synergistic effect, the total amount of drugs administered to a patient can be advantageously reduced, thereby resulting in a decrease in side effects.

The combination therapy of the invention permits localized reduction in TGFβ in a tumor microenvironment by capturing the TGFβ using a soluble cytokine receptor (TGFβRII) tethered to an antibody moiety targeting a cellular immune checkpoint receptor found on the exterior surface of certain tumor cells or immune cells. An example of an antibody moiety of the invention is to an immune checkpoint protein is anti-PD-L1. This bifunctional molecule, sometimes referred to in this document as an “antibody-cytokine trap,” is effective precisely because the anti-receptor antibody and cytokine trap are physically linked. The resulting advantage (over, for example, administration of the antibody and the receptor as separate molecules) is partly because cytokines function predominantly in the local environment through autocrine and paracrine functions. The antibody moiety directs the cytokine trap to the tumor microenvironment where it can be most effective, by neutralizing the local immunosuppressive autocrine or paracrine effects. Furthermore, in cases where the target of the antibody is internalized upon antibody binding, an effective mechanism for clearance of the cytokine/cytokine receptor complex is provided. Antibody-mediated target internalization has been shown for PD-L1. This is a distinct advantage over using an anti-TGFβ antibody because first, an anti-TGFβ antibody might not be completely neutralizing; and second, the antibody can act as a carrier extending the half-life of the cytokine, and antibody/cytokine complexes often act as a circulating sink that builds up and ultimately dissociates to release the cytokine back in circulation (Montero-Julian et al., Blood. 1995; 85:917-24). The use of a cytokine trap to neutralize the ligand can also be a better strategy than blockading the receptor with an antibody, as in the case of CSF-1. Because CSF-1 is cleared from the circulation by receptor-mediated endocytosis, an anti-CSF-1 receptor antibody blockade caused a significant increase in circulating CSF-1 concentration (Hume et al., Blood. 2012; 119:1810-20)

As described below, treatment with the anti-PD-L1/TGFβ Trap, in combination with at least one additional anti-cancer therapeutic, elicits a synergistic anti-tumor effect due to the simultaneous blockade of the interaction between PD-L1 on tumor cells and PD-1 on immune cells, the neutralization of TGFβ in the tumor microenvironment, and the therapeutic effect of the anti-cancer agent. Without being bound by theory, this presumably is due to a synergistic effect obtained from simultaneous blocking the two major immune escape mechanisms, and in addition, the targeted depletion of the TGFβ in the tumor microenvironment by a single molecular entity, as well as the anti-tumor effect of the additional anti-cancer agent(s). This depletion is achieved by (1) anti-PD-L1 targeting of tumor cells; (2) binding of the TGFβ autocrine/paracrine in the tumor microenvironment by the TGFβ Trap; and (3) destruction of the bound TGFβ through the PD-L1 receptor-mediated endocytosis. The aforementioned mechanisms of action cannot be achieved by the combination therapy of the single agent anti-PD-L1, a TGFβ Trap and additional anti-cancer therapeutics. Furthermore, the TGFβRII fused to the C-terminus of Fc (fragment of crystallization of IgG) was several-fold more potent than the TGFβRII-Fc that places the TGFβRII at the N-terminus of Fc. The superb efficacy obtained with anti-PDL1/TGFβ Trap also allays some concerns that the TGFβRII does not trap TGFβ2. As pointed out by Yang et al., Trends Immunol. 2010; 31:220-227, although some tumor types do secrete TGFβ2 initially, as the tumor progresses, the TGFβ in the tumor microenvironment is predominantly secreted by myeloid-derived suppressor cells, which secrete TGFβ1. In addition to showing great promise as an effective immuno-oncology therapeutic, treatment with soluble TGFβRII can potentially reduce the cardiotoxicity concerns of TGFβ targeting therapies, especially the TGFβRI kinase inhibitors. This is because of the important roles TGFβ2 plays in embryonic development of the heart as well as in repair of myocardial damage after ischemia and reperfusion injury (Roberts et al., J Clin Invest. 1992; 90:2056-62).

### TGFβ as a Cancer Target

TGFβ had been a somewhat questionable target in cancer immunotherapy because of its paradoxical roles as the molecular Jekyll and Hyde of cancer (Bierie et al., Nat Rev Cancer. 2006; 6:506-20). Like some other cytokines, TGFβ activity is developmental stage and context dependent. Indeed TGFβ can act as either a tumor promoter or a tumor suppressor, affecting tumor initiation, progression and metastasis. The mechanisms underlying this dual role of TGFβ remain unclear (Yang et al., Trends Immunol. 2010; 31:220-227). Although it has been postulated that Smad-dependent signaling mediates the growth inhibition of TGFβ signaling, while the Smad independent pathways contribute to its tumor-promoting effect, there are also data showing that the Smad-dependent pathways are involved in tumor progression (Yang et al., Cancer Res. 2008; 68:9107-11).

Both the TGFβ ligand and the receptor have been studied intensively as therapeutic targets. There are three ligand isoforms, TGFβ1, 2 and 3, all of which exist as homodimers. There are also three TGFβ receptors (TGFβR), which are called TGFβR type I, II and III (Lopez-Casillas et al., J Cell Biol. 1994; 124:557-68). TGFβRI is the signaling chain and cannot bind ligand. TGFβRII binds the ligand TGFβ1 and 3, but not TGFβ2, with high affinity. The TGFβ/TGFβ complex recruits TGFβRI to form the signaling complex (Won et al., Cancer Res. 1999; 59:1273-7). TGFβRIII is a positive regulator of TGFβ binding to its signaling receptors and binds all 3 TGFβ isoforms with high affinity. On the cell surface, the TGFβ/TGFβRIII complex binds TGFβRII and then recruits TGFβRI, which displaces TGFβRIII to form the signaling complex.

Although the three different TGFβ isoforms all signal through the same receptor, they are known to have differential expression patterns and non-overlapping functions in vivo. The three different TGF-β isoform knockout mice have distinct phenotypes, indicating numerous non-compensated functions (Bujak et al., Cardiovasc Res. 2007; 74:184-95). While TGFβ1 null mice have hematopoiesis and vasculogenesis defects and TGFβ3 null mice display pulmonary development and defective palatogenesis, TGFβ2 null mice show various developmental abnormalities, the most prominent being multiple cardiac deformities (Bartram et al., Circulation. 2001; 103:2745-52; Yamagishi et al., Anat Rec. 2012; 295:257-67). Furthermore, TGFβ is implicated to play a major role in the repair of myocardial damage after ischemia and reperfusion injury. In an adult heart, cardiomyocytes secrete TGFβ, which acts as an autocrine to maintain the spontaneous beating rate. Importantly, 70-85% of the TGFβ secreted by cardiomyocytes is TGFβ2 (Roberts et al., J Clin Invest. 1992; 90:2056-62). In summary, given the predominant roles of TGFβ1 and TGFβ2 in the tumor microenvironment and cardiac physiology, respectively, a therapeutic agent that neutralizes TGFβ1 but not TGFβ2 could provide an optimal therapeutic index by minimizing the cardiotoxicity without compromising the anti-tumor activity. This is consistent with the findings by the present inventors, who observed a lack of toxicity, including cardiotoxicity, for anti-PD-L1/TGFβ Trap in monkeys.

Therapeutic approaches to neutralize TGFβ include using the extracellular domains of TGFβ receptors as soluble receptor traps and neutralizing antibodies. Of the receptor trap approach, soluble TGFβRIII may seem the obvious choice since it binds all the three TGFβ ligands. However, TGFβRIII, which occurs naturally as a 280-330 kD glucosaminoglycan (GAG)-glycoprotein, with extracellular domain of 762 amino acid residues, is a very complex protein for biotherapeutic development. The soluble TGFβRIII devoid of GAG could be produced in insect cells and shown to be a potent TGFβ neutralizing agent (Vilchis-Landeros et al, Biochem J 355:215, 2001). The two separate binding domains (the endoglin-related and the uromodulin-related) of TGFβRIII could be independently expressed, but they were shown to have affinities 20 to 100 times lower than that of the soluble TGFβRIII, and much diminished neutralizing activity (Mendoza et al., Biochemistry. 2009; 48:11755-65). On the other hand, the extracellular domain of TGFβRII is only 136 amino acid residues in length and can be produced as a glycosylated protein of 25-35 kD. The recombinant soluble TGFβRII was further shown to bind TGFβ1 with a K_{D} of 200 pM, which is fairly similar to the K_{D} of 50 pM for the full length TGFβRII on cells (Lin et al., J Biol Chem. 1995; 270:2747-54). Soluble TGFβRII-Fc was tested as an anti-cancer agent and was shown to inhibit established murine malignant mesothelioma growth in a tumor model (Suzuki et al., Clin Cancer Res. 2004; 10:5907-18). Since TGFβRII does not bind TGFβ2, and TGFβRIII binds TGFβ1 and 3 with lower affinity than TGFβRII, a fusion protein of the endoglin domain of TGFβRIII and extracellular domain of TGFβRII was produced in bacteria and was shown to inhibit the signaling of TGFβ1 and 2 in cell based assays more effectively than either TGFβRII or RIII (Verona et al., Protein Eng Des Sel. 2008; 21:463-73). Despite some encouraging anti-tumor activities in tumor models, to our knowledge no TGFβ receptor trap recombinant proteins have been tested in the clinic.

Still another approach to neutralize all three isoforms of the TGFβ ligands is to screen for a pan-neutralizing anti-TGFβ antibody, or an anti-receptor antibody that blocks the receptor from binding to TGFβ1, 2 and 3. GC1008, a human antibody specific for all isoforms of TGFβ, was in a Phase VII study in patients with advanced malignant melanoma or renal cell carcinoma (Morris et al., J Clin Oncol 2008; 26:9028 (Meeting abstract)). Although the treatment was found to be safe and well tolerated, only limited clinical efficacy was observed, and hence it was difficult to interpret the importance of anti-TGFβ therapy without further characterization of the immunological effects (Flavell et al., Nat Rev Immunol. 2010; 10:554-67). There were also TGFβ-isoform-specific antibodies tested in the clinic. Metelimumab, an antibody specific for TGFβ1 was tested in Phase 2 clinical trial as a treatment to prevent excessive post-operative scarring for glaucoma surgery; and Lerdelimumab, an antibody specific for TGFβ2, was found to be safe but ineffective at improving scarring after eye surgery in a Phase 3 study (Khaw et al., Ophthalmology 2007; 114:1822-1830). Anti-TGFβRII antibodies that block the receptor from binding to all three TGFβ isoforms, such as the anti-human TGFβRII antibody TR1 and anti-mouse TGFβRII antibody MT1, have also shown some therapeutic efficacy against primary tumor growth and metastasis in mouse models (Zhong et al., Clin Cancer Res. 2010; 16:1191-205). To date, the vast majority of the studies on TGFβ targeted anticancer treatment, including small molecule inhibitors of TGFβ signaling that often are quite toxic, are mostly in the preclinical stage and the anti-tumor efficacy obtained has been limited (Calone et al., Exp Oncol. 2012; 34:9-16; Connolly et al., Int J Biol Sci. 2012; 8:964-78).

The antibody-TGFβ trap of the invention, for use in the combination therapy of the invention, is a bifunctional protein containing at a least portion of a human TGFβ Receptor II (TGFβRII) that is capable of binding TGFβ. In one embodiment, the TGFβ trap polypeptide is a soluble portion of the human TGFβ Receptor Type 2 Isoform A (SEQ ID NO: 8) that is capable of binding TGFβ. In a further embodiment, TGFβ trap polypeptide contains at least amino acids 73-184 of SEQ ID NO:8. In yet a further embodiment, the TGFβ trap polypeptide contains amino acids 24-184 of SEQ ID NO:8. In another embodiment, the TGFβ trap polypeptide is a soluble portion of the human TGFβ Receptor Type 2 Isoform B (SEQ ID NO: 9) that is capable of binding TGFβ. In a further embodiment, TGFβ trap polypeptide contains at least amino acids 48-159 of SEQ ID NO:9. In yet a further embodiment, the TGFβ trap polypeptide contains amino acids 24-159 of SEQ ID NO:9. In yet a further embodiment, the TGFβ trap polypeptide contains amino acids 24-105 of SEQ ID NO:9.

### Immune Checkpoint Dis-Inhibition

The approach of targeting T cell inhibition checkpoints for dis-inhibition with therapeutic antibodies is an area of intense investigation (for a review, see Pardoll, Nat Rev Cancer. 2012; 12:253-264). In one approach, the antibody moiety or antigen binding fragment thereof targets T cell inhibition checkpoint receptor proteins on the T cell, such as, for example: CTLA-4, PD-1, BTLA, LAG-3, TIM-3, and LAIR1. In another approach, the antibody moiety targets the counter-receptors on antigen presenting cells and tumor cells (which co-opt some of these counter-receptors for their own immune evasion), such as, for example: PD-L1 (B7-H1), B7-DC, HVEM, TIM-4, B7-H3, or B7-H4.

The invention contemplates the use of antibody TGFβ traps that target, through their antibody moiety or antigen binding fragment thereof, T cell inhibition checkpoints for dis-inhibition. To that end the present inventors have tested the anti-tumor efficacy of combining a TGFβ trap with antibodies targeting various T cell inhibition checkpoint receptor proteins, such as anti-PD-1, anti-PD-L1, anti-TIM-3 and anti-LAG3. The present inventors found that combining a TGFβ trap with an anti-PD-L1 antibody exhibited remarkable anti-tumor activity beyond what was observed with the monotherapies. In contrast, none of the other combinations with antibodies to the targets listed above showed any superior efficacy. In particular, one may have expected that a combination treatment of a TGFβ trap with an anti-PD-1 antibody would demonstrate similar activity to the one observed with anti-PD-L1, as PD-1/PD-L1 are cognate receptors that bind to each other to effect the immune checkpoint inhibition. However, this is not what the present inventors have found.

### Anti-PD-L1 Antibodies

The invention can include the use of any anti-PD-L1 antibody, or antigen-binding fragment thereof, described in the art. Anti-PD-L1 antibodies are commercially available, for example, the 29E2A3 antibody (Biolegend, Cat. No. 329701). Antibodies can be monoclonal, chimeric, humanized, or human. Antibody fragments include Fab, F(ab′)2, scFv and Fv fragments, which are described in further detail below.

Exemplary antibodies are described in PCT Publication WO 2013/079174. These antibodies can include a heavy chain variable region polypeptide including an HVR-H1, HVR-H2, and HVR-H3 sequence, where:

(a) the HVR-H1 sequence is
X₁YX₂MX₃;
(b) the HVR-H2 sequence is
(SEQ ID NO: 21)
SIYPSGGX₄TFYADX₅VKG;
(c) the HVR-H3 sequence is
(SEQ ID NO: 22)
IKLGTVTTVX₆Y;

further where: X₁ is K, R, T, Q, G, A, W, M, I, or S; X₂ is V, R, K, L, M, or I; X₃ is H, T, N, Q, A, V, Y, W, F, or M; X₄ is F or I; X₅ is S or T; X₆ is E or D.

In a one embodiment, X₁ is M, I, or S; X₂ is R, K, L, M, or I; X₃ is F or M; X₄ is F or I; X₅ is S or T; X₆ is E or D.

In another embodiment X₁ is M, I, or S; X₂ is L, M, or I; X₃ is F or M; X₄ is I; X₅ is S or T; X₆ is D.

In still another embodiment, X₁ is S; X₂ is I; X₃ is M; X₄ is I; X₅ is T; X₆ is D.

In another aspect, the polypeptide further includes variable region heavy chain framework sequences juxtaposed between the HVRs according to the formula: (HC-FR1)-(HVR-H1)-(HC-FR2)-(HVR-H2)-(HC-FR3)-(HVR-H3)-(HC-FR4).

In yet another aspect, the framework sequences are derived from human consensus framework sequences or human germline framework sequences.

In a still further aspect, at least one of the framework sequences is the following:

HC-FR1 is
(SEQ ID NO: 23)
EVQLLESGGGLVQPGGSLRLSCAASGFTFS;
HC-FR2 is
(SEQ ID NO: 24)
WVRQAPGKGLEWVS;
HC-FR3 is
(SEQ ID NO: 25)
RFTISRDNSKNTLYLQMNSLRAEDTAVYYCAR;
HC-FR4 is
(SEQ ID NO: 26)
WGQGTLVTVSS.

In a still further aspect, the heavy chain polypeptide is further combined with a variable region light chain including an HVR-L1, HVR-L2, and HVR-L3, where:

(a) the HVR-L1 sequence is
(SEQ ID NO: 27)
TGTX₇X₈DVGX₉YNYVS;
(b) the HVR-L2 sequence is
(SEQ ID NO: 28)
X₁₀VX₁₁X₁₂RPS;
(c) the HVR-L3 sequence is
(SEQ ID NO: 29)
SSX₁₃TX₁₄X₁₅X₁₆X₁₇RV;

further where: X₇ is N or S; X₈ is T, R, or S; X₉ is A or G; X₁₀ is E or D; X₁₁ is I, N or S; X₁₂ is D, H or N; X₁₃ is F or Y; X₁₄ is N or S; X₁₅ is R, T or S; X₁₆ is G or S; X₁₇ is I or T.

In another embodiment, X₇ is N or S; X₈ is T, R, or S; X₉ is A or G; X₁₀ is E or D; X₁₁ is N or S; X₁₂ is N; X₁₃ is F or Y; X₁₄ is S; X₁₅ is S; X₁₆ is G or S; X₁₇ is T.

In still another embodiment, X₇ is S; X₈ is S; X₉ is G; X₁₀ is D; X₁₁ is S; X₁₂ is N; X₁₃ is Y; X₁₄ is S; X₁₅ is S; X₁₆ is S; X₁₇ is T.

In a still further aspect, the light chain further includes variable region light chain framework sequences juxtaposed between the HVRs according to the formula: (LC-FR1)-(HVR-L1)-(LC-FR2)-(HVR-L2)-(LC-FR3)-(HVR-L3)-(LC-FR4).

In a still further aspect, the light chain framework sequences are derived from human consensus framework sequences or human germline framework sequences.

In a still further aspect, the light chain framework sequences are lambda light chain sequences.

In a still further aspect, at least one of the framework sequence is the following:

LC-FR1 is
(SEQ ID NO: 30)
QSALTQPASVSGSPGQSITISC;
LC-FR2 is
(SEQ ID NO: 31)
WYQQHPGKAPKLMIY;
LC-FR3 is
(SEQ ID NO: 32)
GVSNRFSGSKSGNTASLTISGLQAEDEADYYC;
LC-FR4 is
(SEQ ID NO: 33)
FGTGTKVTVL.

In another embodiment, the invention provides an anti-PD-L1 antibody or antigen binding fragment including a heavy chain and a light chain variable region sequence, where:

(a) the heavy chain includes an HVR-H1, HVR-H2, and HVR-H3, wherein further: (i) the HVR-H1 sequence is X₁YX₂MX₃; (ii) the HVR-H2 sequence is SIYPSGGX₄TFYADX₅VKG (SEQ ID NO:21); (iii) the HVR-H3 sequence is IKLGTVTTVX₆Y (SEQ ID NO:22), and;

(b) the light chain includes an HVR-L1, HVR-L2, and HVR-L3, wherein further: (iv) the HVR-L1 sequence is TGTX₇X₈DVGX₉YNYVS (SEQ ID NO:27); (v) the HVR-L2 sequence is X₁₀VX₁₁X₁₂RPS (SEQ ID NO:28); (vi) the HVR-L3 sequence is SSX₁₃TX₁₄X₁₅X₁₆X₁₇RV (SEQ ID NO:29); wherein: X₁ is K, R, T, Q, G, A, W, M, I, or S; X₂ is V, R, K, L, M, or I; X₃ is H, T, N, Q, A, V, Y, W, F, or M; X₄ is F or I; X₅ is S or T; X₆ is E or D; X₇ is N or S; X₈ is T, R, or S; X₉ is A or G; X₁₀ is E or D; X₁₁ is I, N, or S; X₁₂ is D, H, or N; X₁₃ is F or Y; X₁₄ is N or S; X₁₅ is R, T, or S; X₁₆ is G or S; X₁₇ is I or T.

In one embodiment, X₁ is M, I, or S; X₂ is R, K, L, M, or I; X₃ is F or M; X₄ is F or I; X₅ is S or T; X₆ is E or D; X₇ is N or S; X₈ is T, R, or S; X₉ is A or G; X₁₀ is E or D; X₁₁ is N or S; X₁₂ is N; X₁₃ is F or Y; X₁₄ is S; X₁₅ is S; X₁₆ is G or S; X₁₇ is T.

In another embodiment, X₁ is M, I, or S; X₂ is L, M, or I; X₃ is F or M; X₄ is I; X₅ is S or T; X₆ is D; X₇ is N or S; X₈ is T, R, or S; X₉ is A or G; X₁₀ is E or D; X₁₁ is N or S; X₁₂ is N; X₁₃ is F or Y; X₁₄ is S; X₁₅ is S; X₁₆ is G or S; X₁₇ is T.

In still another embodiment, X₁ is S; X₂ is I; X₃ is M; X₄ is I; X₅ is T; X₆ is D; X₇ is S; X₈ is S; X₉ is G; X₁₀ is D; X₁₁ is S; X₁₂ is N; X₁₃ is Y; X₁₄ is S; X₁₅ is S; X₁₆ is S; X₁₇ is T.

In a further aspect, the heavy chain variable region includes one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions include one or more framework sequences juxtaposed between the HVRs as: (LC-FR1 MHVR-L1)-(LC-FR2)-(HVR-L2)-(LC-FR3)-(HVR-L3)-(LC-FR4).

In a still further aspect, the framework sequences are derived from human consensus framework sequences or human germline sequences.

In a still further aspect, one or more of the heavy chain framework sequences is the following:

HC-FR1 is
(SEQ ID NO: 23)
EVQLLESGGGLVQPGGSLRLSCAASGFTFS;
HC-FR2 is
(SEQ ID NO: 24)
WVRQAPGKGLEWVS;
HC-FR3 is
(SEQ ID NO: 25)
RFTISRDNSKNTLYLQMNSLRAEDTAVYYCAR;
HC-FR4 is
(SEQ ID NO: 26)
WGQGTLVTVSS.

In a still further aspect, the light chain framework sequences are lambda light chain sequences.

In a still further aspect, one or more of the light chain framework sequences is the following:

LC-FR1 is
(SEQ ID NO: 30)
QSALTQPASVSGSPGQSITISC;
LC-FR2 is
(SEQ ID NO: 31)
WYQQHPGKAPKLMIY;
LC-FR3 is
(SEQ ID NO: 32)
GVSNRFSGSKSGNTASLTISGLQAEDEADYYC;
LC-FR4 is
(SEQ ID NO: 33)
FGTGTKVTVL.

In a still further aspect, the heavy chain variable region polypeptide, antibody, or antibody fragment further includes at least a C_{H}1 domain.

In a more specific aspect, the heavy chain variable region polypeptide, antibody, or antibody fragment further includes a C_{H}1, a C_{H}2, and a C_{H}3 domain.

In a still further aspect, the variable region light chain, antibody, or antibody fragment further includes a C_{L} domain.

In a still further aspect, the antibody further includes a C_{H}1, a C_{H}2, a C_{H}3, and a C_{L} domain.

In a still further specific aspect, the antibody further includes a human or murine constant region.

In a still further aspect, the human constant region is selected from the group consisting of IgG1, IgG2, IgG2, IgG3, and IgG4.

In a still further specific aspect, the human or murine constant region is lgG1.

In yet another embodiment, the invention features an anti-PD-L1 antibody including a heavy chain and a light chain variable region sequence, where:

(a) the heavy chain includes an HVR-H1, an HVR-H2, and an HVR-H3, having at least 80% overall sequence identity to SYIMM (SEQ ID NO:34), SIYPSGGITFYADTVKG (SEQ ID NO:35), and IKLGTVTTVDY (SEQ ID NO:36), respectively, and

(b) the light chain includes an HVR-L1, an HVR-L2, and an HVR-L3, having at least 80% overall sequence identity to TGTSSDVGGYNYVS (SEQ ID NO:37), DVSNRPS (SEQ ID NO:38), and SSYTSSSTRV (SEQ ID NO:39), respectively.

In a specific aspect, the sequence identity is 81%, 82%, 83%, 84%, 85%, 86%, 87%, 88%, 89%, 90%, 91%, 92%, 93%, 94%, 95%, 96%, 97%, 98%, 99%, or 100%.

In yet another embodiment, the invention features an anti-PD-L1 antibody including a heavy chain and a light chain variable region sequence, where:

(a) the heavy chain includes an HVR-H1, an HVR-H2, and an HVR-H3, having at least 80% overall sequence identity to MYMMM (SEQ ID NO:40), SIYPSGGITFYADSVKG (SEQ ID NO:41), and IKLGTVTTVDY (SEQ ID NO:36), respectively, and

(b) the light chain includes an HVR-L1, an HVR-L2, and an HVR-L3, having at least 80% overall sequence identity to TGTSSDVGAYNYVS (SEQ ID NO:42), DVSNRPS (SEQ ID NO:38), and SSYTSSSTRV (SEQ ID NO:39), respectively.

In a specific aspect, the sequence identity is 81%, 82%, 83%, 84%, 85%, 86%, 87%, 88%, 89%, 90%, 91%, 92%, 93%, 94%, 95%, 96%, 97%, 98%, 99%, or 100%.

In a still further aspect, in the antibody or antibody fragment according to the invention, as compared to the sequences of HVR-H1, HVR-H2, and HVR-H3, at least those amino acids remain unchanged that are highlighted by underlining as follows:

(a) in HVR-H1
(SEQ ID NO: 34)
SYIMM,
(b) in HVR-H2
(SEQ ID NO: 35)
SIYPSGGITFYADTVKG,
(c) in HVR-H3
(SEQ ID NO: 36)
IKLGTVTTVDY;

and further where, as compared to the sequences of HVR-L1, HVR-L2, and HVR-L3 at least those amino acids remain unchanged that are highlighted by underlining as follows:

(a) HVR-L1
(SEQ ID NO: 37)
TGTSSDVGGYNYVS
(b) HVR-L2
(SEQ ID NO: 38)
DVSNRPS
(c) HVR-L3
(SEQ ID NO: 39)
SSYTSSSTRV.

In another aspect, the heavy chain variable region includes one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions include one or more framework sequences juxtaposed between the HVRs as: (LC-FR1)-(HVR-L1)-(LC-FR2)-(HVR-L2)-(LC-FR3)-(HVR-L3)-(LC-FR4).

In yet another aspect, the framework sequences are derived from human germline sequences.

In a still further aspect, one or more of the heavy chain framework sequences is the following:

HC-FR1 is
(SEQ ID NO: 23)
EVQLLESGGGLVQPGGSLRLSCAASGFTFS;
HC-FR2 is
(SEQ ID NO: 24)
WVRQAPGKGLEWVS;
HC-FR3 is
(SEQ ID NO: 25)
RFTISRDNSKNTLYLQMNSLRAEDTAVYYCAR;
HC-FR4 is
(SEQ ID NO: 26)
WGQGTLVTVSS.

In a still further aspect, the light chain framework sequences are derived from a lambda light chain sequence.

In a still further aspect, one or more of the light chain framework sequences is the following:

LC-FR1 is
(SEQ ID NO: 30)
QSALTQPASVSGSPGQSITISC;
LC-FR2 is
(SEQ ID NO: 31)
WYQQHPGKAPKLMIY;
LC-FR3 is
(SEQ ID NO: 32)
GVSNRFSGSKSGNTASLTISGLQAEDEADYYC;
LC-FR4 is
(SEQ ID NO: 33)
FGTGTKVTVL.

In a still further specific aspect, the antibody further includes a human or murine constant region.

In a still further aspect, the human constant region is selected from the group consisting of IgG1, IgG2, IgG2, IgG3, and IgG4.

In a still further embodiment, the invention features an anti-PD-L1 antibody including a heavy chain and a light chain variable region sequence, where:

(a) the heavy chain sequence has at least 85%
sequence identity to the heavy chain sequence:
(SEQ ID NO: 43)
EVQLLESGGGLVQPGGSLRLSCAASGFTFSSYIMMVWRQAPGKGLEW
VSSIYPSGGITFYADWKGRFTISRDNSKNTLYLQMNSLRAEDTAVYY
CARIKLGTVTTVDYWGQGTLVTVSS,
and
(b) the light chain sequence has at least 85%
sequence identity to the light chain sequence:
(SEQ ID NO: 44)
QSALTQPASVSGSPGQSITISCTGTSSDVGGYNYVSWYQQHPGKAPK
LMIYDVSNRPSGVSNRFSGSKSGNTASLTISGLQAEDEADYYCSSYT
SSSTRVFGTGTKVTVL.

In a specific aspect, the sequence identity is 86%, 87%, 88%, 89%, 90%, 91%, 92%, 93%, 94%, 95%, 96%, 97%, 98%, 99%, or 100%.

In a still further embodiment, the invention provides for an anti-PD-L1 antibody including a heavy chain and a light chain variable region sequence, where:

(a) the heavy chain sequence has at least 85%
sequence identity to the heavy chain sequence:
(SEQ ID NO: 45)
EVQLLESGGGLVQPGGSLRLSCAASGFTFSMYMMMWVRQAPGKGLEV
WSSIYPSGGITFYADSVKGRFTISRDNSKNTLYLQMNSLRAEDTAIY
YCARIKLGTVTTVDYWGQGTLVTVSS,
and
(b) the light chain sequence has at least 85%
sequence identity to the light chain sequence:
(SEQ ID NO: 46)
QSALTQPASVSGSPGQSITISCTGTSSDVGAYNYVSWYQQHPGKAPK
LMIYDVSNRPSGVSNRFSGSKSGNTASLTISGLQAEDEADYYCSSYT
SSSTRVFGTGTKVTVL.

In a specific aspect, the sequence identity is 86%, 87%, 88%, 89%, 90%, 91%, 92%, 93%, 94%, 95%, 96%, 97%, 98%, 99% or 100%. In another embodiment the antibody binds to human, mouse, or cynomolgus monkey PD-L1. In a specific aspect the antibody is capable of blocking the interaction between human, mouse, or cynomolgus monkey PD-L1 and the respective human, mouse, or cynomolgus monkey PD-1 receptors.

In another embodiment, the antibody binds to human PD-L1 with a K_{D} of 5×10⁻⁹ M or less, preferably with a K_{D} of 2×10⁻⁹M or less, and even more preferred with a K_{D} of 1×10⁻⁹ M or less.

In yet another embodiment, the invention relates to an anti-PD-L1 antibody or antigen binding fragment thereof which binds to a functional epitope including residues Y56 and D61 of human PD-L1.

In a specific aspect, the functional epitope further includes E58, E60, Q66, R113, and M115 of human PD-L1.

In a more specific aspect, the antibody binds to a conformational epitope, including residues 54-66 and 112-122 of human PD-L1.

In a further embodiment, the invention is related to the use of an anti-PD-L1 antibody, or antigen binding fragment thereof, which cross-competes for binding to PD-L1 with an antibody according to the invention as described herein.

In a still further embodiment, the invention features proteins and polypeptides including any of the above described anti-PD-L1 antibodies in combination with at least one pharmaceutically acceptable carrier for use in the combination therapy of the invention.

In a still further embodiment, the invention features the use of an isolated nucleic acid encoding a polypeptide, or light chain or a heavy chain variable region sequence of an anti-PD-L1 antibody, or antigen binding fragment thereof, as described herein. In a still further embodiment, the invention provides for an isolated nucleic acid encoding a light chain or a heavy chain variable region sequence of an anti-PD-L1 antibody, wherein:

(a) the heavy chain includes an HVR-H1, an HVR-H2, and an HVR-H3 sequence having at least 80% sequence identity to SYIMM (SEQ ID NO: 34), SIYPSGGITFYADTVKG (SEQ ID NO: 35), and IKLGTVTTVDY (SEQ ID NO: 36), respectively, or

(b) the light chain includes an HVR-L1, an HVR-L2, and an HVR-L3 sequence having at least 80% sequence identity to TGTSSDVGGYNYVS (SEQ ID NO: 37), DVSNRPS (SEQ ID NO: 38), and SSYTSSSTRV (SEQ ID NO: 39), respectively.

In a specific aspect, the sequence identity is 81%, 82%, 83%, 84%, 85%, 86%, 87%, 88%, 89%, 90%, 91%, 92%, 93%, 94%, 95%, 96%, 97%, 98%, 99%, or 100%.

| In a further aspect, the nucleic acid sequence for the heavy chain | |
|---|---|
| is (SEQ ID NO: 47): | |
| atggagttgc ctgttaggct gttggtgctg atgttctgga ttcctgctag ctccagcgag | 60 |
| gtgcagctgc tggaatccgg cggaggactg gtgcagcctg gcggctccct gagactgtct | 120 |
| tgcgccgcct ccggcttcac cttctccagc tacatcatga tgtgggtgcg acaggcccct | 180 |
| ggcaagggcc tggaatgggt gtcctccatc tacccctccg gcggcatcac cttctacgcc | 240 |
| gacaccgtga agggccggtt caccatctcc cgggacaact ccaagaacac cctgtacctg | 300 |
| cagatgaact ccctgcgggc cgaggacacc gccgtgtact actgcgcccg gatcaagctg | 360 |
| ggcaccgtga ccaccgtgga ctactggggc cagggcaccc tggtgacagt gtcctccgcc | 420 |
| tccaccaagg gcccatcggt cttccccctg gcaccctcct ccaagagcac ctctgggggc | 480 |
| acagcggccc tgggctgcct ggtcaaggac tacttccccg aaccggtgac ggtgtcgtgg | 540 |
| aactcaggcg ccctgaccag cggcgtgcac accttcccgg ctgtcctaca gtcctcagga | 600 |
| ctctactccc tcagcagcgt ggtgaccgtg ccctccagca gcttgggcac ccagacctac | 660 |
| atctgcaacg tgaatcacaa gcccagcaac accaaggtgg acaagaaagt tgagcccaaa | 720 |
| tcttgtgaca aaactcacac atgcccaccg tgcccagcac ctgaactcct ggggggaccg | 780 |
| tcagtcttcc tcttcccccc aaaacccaag gacaccctca tgatctcccg gacccctgag | 840 |
| gtcacatgcg tggtggtgga cgtgagccac gaagaccctg aggtcaagtt caactggtac | 900 |
| gtggacggcg tggaggtgca taatgccaag acaaagccgc gggaggagca gtacaacagc | 960 |
| acgtaccgtg tggtcagcgt cctcaccgtc ctgcaccagg actggctgaa tggcaaggag | 1020 |
| tacaagtgca aggtctccaa caaagccctc ccagccccca tcgagaaaac catctccaaa | 1080 |
| gccaaagggc agccccgaga accacaggtg tacaccctgc ccccatcacg ggatgagctg | 1140 |
| accaagaacc aggtcagcct gacctgcctg gtcaaaggct tctatcccag cgacatcgcc | 1200 |
| gtggagtggg agagcaatgg gcagccggag aacaactaca agaccacgcc tcccgtgctg | 1260 |
| gactccgacg gctccttctt cctctatagc aagctcaccg tggacaagag caggtggcag | 1320 |
| caggggaacg tcttctcatg ctccgtgatg catgaggctc tgcacaacca ctacacgcag | 1380 |
| aagagcctct ccctgtcccc gggtaaa | 1407 |
| and the nucleic acid sequence for the light chain is (SEQ ID NO: 48): | |
| atggagttgc ctgttaggct gttggtgctg atgttctgga ttcctgcttc cttaagccag | 60 |
| tccgccctga cccagcctgc ctccgtgtct ggctcccctg gccagtccat caccatcagc | 120 |
| tgcaccggca cctccagcga cgtgggcggc tacaactacg tgtcctggta tcagcagcac | 180 |
| cccggcaagg cccccaagct gatgatctac gacgtgtcca accggccctc cggcgtgtcc | 240 |
| aacagattct ccggctccaa gtccggcaac accgcctccc tgaccatcag cggactgcag | 300 |
| gcagaggacg aggccgacta ctactgctcc tcctacacct cctccagcac cagagtgttc | 360 |
| ggcaccggca caaaagtgac cgtgctgggc cagcccaagg ccaacccaac cgtgacactg | 420 |
| ttccccccat cctccgagga actgcaggcc aacaaggcca ccctggtctg cctgatctca | 480 |
| gatttctatc caggcgccgt gaccgtggcc tggaaggctg atggctcccc agtgaaggcc | 540 |
| ggcgtggaaa ccaccaagcc ctccaagcag tccaacaaca aatacgccgc ctcctcctac | 600 |
| ctgtccctga cccccgagca gtggaagtcc caccggtcct acagctgcca ggtcacacac | 660 |
| gagggctcca ccgtggaaaa gaccgtcgcc cccaccgagt gctca | 705 |

Further exemplary anti-PD-L1 antibodies that can be used in an anti-PD-L1/TGFβ Trap are described in US patent application publication US 2010/0203056. In one embodiment of the invention, the antibody moiety is YW243.55S70. In another embodiment of the invention, the antibody moiety is MPDL3280A.

In a further embodiment, the invention features the use of an anti-PD-L1 antibody moiety including a heavy chain and a light chain variable region sequence, where:

(a) the heavy chain sequence has at least 85%
sequence identity to the heavy chain sequence:
(SEQ ID NO: 12)
EVQLVESGGGLVQPGGSLRLSCAASGFTFSDSWIHWVRQAPGKGLEW
VAWISPYGGSTYYADSVKGRFTISADTSKNTAYLQMNSLRAEDTAVY
YCARRHWPGGFDYWGQGTLVTVSS,
and
(b) the light chain sequence has at least 85%
sequence identity to the light chain sequence:
(SEQ ID NO: 13)
DIQMTQSPSSLSASVGDRVTITCRASQDVSTAVAWYQQKPGKAPKLL
IYSASFLYSGVPSRFSGSGSGTDFTLTISSLQPEDFATYYCQQYLYH
PATFGQGTKVEIKR.

In a specific aspect, the sequence identity is 86%, 87%, 88%, 89%, 90%, 91%, 92%, 93%, 94%, 95%, 96%, 97%, 98%, or 99%.

In a further embodiment, the invention features the use of an anti-PD-L1 antibody moiety including a heavy chain and a light chain variable region sequence, where:

(a) the heavy chain variable region sequence is:
(SEQ ID NO: 12)
EVQLVESGGGLVQPGGSLRLSCAASGFTFSDSWIHWVRQAPGKGLEW
VAWISPYGGSTYYADSVKGRFTISADTSKNTAYLQMNSLRAEDTAVY
YCARRHWPGGFDYWGQGTLVTVSS,
and
(b) the light chain variable region sequence is:
(SEQ ID NO: 13)
DIQMTQSPSSLSASVGDRVTITCRASQDVSTAVAWYQQKPGKAPKLL
IYSASFLYSGVPSRFSGSGSGTDFTLTISSLQPEDFATYYCQQYLYH
PATFGQGTKVEIKR.

In a further embodiment, the invention features an anti-PD-L1 antibody moiety including a heavy chain and a light chain variable region sequence, where:

(a) the heavy chain variable region sequence is:
(SEQ ID NO: 14)
EVQLVESGGGLVQPGGSLRLSCAASGFTFSDSWIHWVRQAPGKGLEW
VAWISPYGGSTYYADSVKGRFTISADTSKNTAYLQMNSLRAEDTAVY
YCARRHWPGGFDYWGQGTLVTVSA,
and
(b) the light chain variable region sequence is:
(SEQ ID NO: 13)
DIQMTQSPSSLSASVGDRVTITCRASQDVSTAVAWYQQKPGKAPKLL
IYSASFLYSGVPSRFSGSGSGTDFTLTISSLQPEDFATYYCQQYLYH
PATFGQGTKVEIKR.

Yet further exemplary anti-PD-L1 antibodies that can be used in an anti-PD-L1/TGFβ Trap are described in US patent publication U.S. Pat. No. 7,943,743.

In one embodiment of the invention, the anti-PD-L1 antibody is MDX-1105.

In yet a further embodiment, the anti-PD-L1 antibody is MEDI-4736.

### Constant Region

The proteins and peptides for use in the combination therapy of the invention can include a constant region of an immunoglobulin or a fragment, analog, variant, mutant, or derivative of the constant region. In preferred embodiments, the constant region is derived from a human immunoglobulin heavy chain, for example, IgG1, IgG2, IgG3, IgG4, or other classes. In one embodiment, the constant region includes a CH2 domain. In another embodiment, the constant region includes CH2 and CH3 domains or includes hinge-CH2-CH3. Alternatively, the constant region can include all or a portion of the hinge region, the CH2 domain and/or the CH3 domain.

In one embodiment, the constant region contains a mutation that reduces affinity for an Fc receptor or reduces Fc effector function. For example, the constant region can contain a mutation that eliminates the glycosylation site within the constant region of an IgG heavy chain. In some embodiments, the constant region contains mutations, deletions, or insertions at an amino acid position corresponding to Leu234, Leu235, Gly236, Gly237, Asn297, or Pro331 of IgG1 (amino acids are numbered according to EU nomenclature). In a particular embodiment, the constant region contains a mutation at an amino acid position corresponding to Asn297 of IgG1. In alternative embodiments, the constant region contains mutations, deletions, or insertions at an amino acid position corresponding to Leu281, Leu282, Gly283, Gly284, Asn344, or Pro378 of IgG1.

In some embodiments, the constant region contains a CH2 domain derived from a human IgG2 or IgG4 heavy chain. Preferably, the CH2 domain contains a mutation that eliminates the glycosylation site within the CH2 domain. In one embodiment, the mutation alters the asparagine within the Gln-Phe-Asn-Ser (SEQ ID NO: 15) amino acid sequence within the CH2 domain of the IgG2 or IgG4 heavy chain. Preferably, the mutation changes the asparagine to a glutamine. Alternatively, the mutation alters both the phenylalanine and the asparagine within the Gln-Phe-Asn-Ser (SEQ ID NO: 15) amino acid sequence. In one embodiment, the Gln-Phe-Asn-Ser (SEQ ID NO: 15) amino acid sequence is replaced with a Gln-Ala-Gln-Ser (SEQ ID NO: 16) amino acid sequence. The asparagine within the Gln-Phe-Asn-Ser (SEQ ID NO: 15) amino acid sequence corresponds to Asn297 of IgG1.

In another embodiment, the constant region includes a CH2 domain and at least a portion of a hinge region. The hinge region can be derived from an immunoglobulin heavy chain, e.g., IgG1, IgG2, IgG3, IgG4, or other classes. Preferably, the hinge region is derived from human IgG1, IgG2, IgG3, IgG4, or other suitable classes. More preferably the hinge region is derived from a human IgG1 heavy chain. In one embodiment the cysteine in the Pro-Lys-Ser-Cys-Asp-Lys (SEQ ID NO: 17) amino acid sequence of the IgG1 hinge region is altered. In a preferred embodiment the Pro-Lys-Ser-Cys-Asp-Lys (SEQ ID NO: 17) amino acid sequence is replaced with a Pro-Lys-Ser-Ser-Asp-Lys (SEQ ID NO: 18) amino acid sequence. In one embodiment, the constant region includes a CH2 domain derived from a first antibody isotype and a hinge region derived from a second antibody isotype. In a specific embodiment, the CH2 domain is derived from a human IgG2 or IgG4 heavy chain, while the hinge region is derived from an altered human IgG1 heavy chain.

The alteration of amino acids near the junction of the Fc portion and the non-Fc portion can dramatically increase the serum half-life of the Fc fusion protein (PCT publication WO 01/58957, the disclosure of which is hereby incorporated by reference). Accordingly, the junction region of a protein or polypeptide of the present invention can contain alterations that, relative to the naturally-occurring sequences of an immunoglobulin heavy chain and erythropoietin, preferably lie within about 10 amino acids of the junction point. These amino acid changes can cause an increase in hydrophobicity. In one embodiment, the constant region is derived from an IgG sequence in which the C-terminal lysine residue is replaced. Preferably, the C-terminal lysine of an IgG sequence is replaced with a non-lysine amino acid, such as alanine or leucine, to further increase serum half-life. In another embodiment, the constant region is derived from an IgG sequence in which the Leu-Ser-Leu-Ser (SEQ ID NO: 19) amino acid sequence near the C-terminus of the constant region is altered to eliminate potential junctional T-cell epitopes. For example, in one embodiment, the Leu-Ser-Leu-Ser (SEQ ID NO: 19) amino acid sequence is replaced with an Ala-Thr-Ala-Thr (SEQ ID NO: 20) amino acid sequence. In other embodiments, the amino acids within the Leu-Ser-Leu-Ser (SEQ ID NO: 19) segment are replaced with other amino acids such as glycine or proline. Detailed methods of generating amino acid substitutions of the Leu-Ser-Leu-Ser (SEQ ID NO: 19) segment near the C-terminus of an IgG1, IgG2, IgG3, IgG4, or other immunoglobulin class molecule have been described in U.S. Patent Publication No. 2003/0166877, the disclosure of which is hereby incorporated by reference.

Suitable hinge regions for the present invention can be derived from IgG1, IgG2, IgG3, IgG4, and other immunoglobulin classes. The IgG1 hinge region has three cysteines, two of which are involved in disulfide bonds between the two heavy chains of the immunoglobulin. These same cysteines permit efficient and consistent disulfide bonding formation between Fc portions. Therefore, a preferred hinge region of the present invention is derived from IgG1, more preferably from human IgG1. In some embodiments, the first cysteine within the human IgG1 hinge region is mutated to another amino acid, preferably serine. The IgG2 isotype hinge region has four disulfide bonds that tend to promote oligomerization and possibly incorrect disulfide bonding during secretion in recombinant systems. A suitable hinge region can be derived from an IgG2 hinge; the first two cysteines are each preferably mutated to another amino acid. The hinge region of IgG4 is known to form interchain disulfide bonds inefficiently. However, a suitable hinge region for the present invention can be derived from the IgG4 hinge region, preferably containing a mutation that enhances correct formation of disulfide bonds between heavy chain-derived moieties (Angal S, et al. (1993) Mol. Immunol., 30:105-8).

In accordance with the present invention, the constant region can contain CH2 and/or CH3 domains and a hinge region that are derived from different antibody isotypes, i.e., a hybrid constant region. For example, in one embodiment, the constant region contains CH2 and/or CH3 domains derived from IgG2 or IgG4 and a mutant hinge region derived from IgG1. Alternatively, a mutant hinge region from another IgG subclass is used in a hybrid constant region. For example, a mutant form of the IgG4 hinge that allows efficient disulfide bonding between the two heavy chains can be used. A mutant hinge can also be derived from an IgG2 hinge in which the first two cysteines are each mutated to another amino acid. Assembly of such hybrid constant regions has been described in U.S. Patent Publication No. 2003/0044423, the disclosure of which is hereby incorporated by reference.

In accordance with the present invention, the constant region can contain one or more mutations described herein. The combinations of mutations in the Fc portion can have additive or synergistic effects on the prolonged serum half-life and increased in vivo potency of the bifunctional molecule. Thus, in one exemplary embodiment, the constant region can contain (i) a region derived from an IgG sequence in which the Leu-Ser-Leu-Ser (SEQ ID NO: 19) amino acid sequence is replaced with an Ala-Thr-Ala-Thr (SEQ ID NO: 20) amino acid sequence; (ii) a C-terminal alanine residue instead of lysine; (iii) a CH2 domain and a hinge region that are derived from different antibody isotypes, for example, an IgG2 CH2 domain and an altered IgG1 hinge region; and (iv) a mutation that eliminates the glycosylation site within the IgG2-derived CH2 domain, for example, a Gln-Ala-Gln-Ser (SEQ ID NO: 16) amino acid sequence instead of the Gln-Phe-Asn-Ser (SEQ ID NO: 15) amino acid sequence within the IgG2-derived CH2 domain.

### Antibody Fragments

The proteins and polypeptides of the invention for use in the combination therapy of the invention can also include antigen-binding fragments of antibodies. Exemplary antibody fragments include scFv, Fv, Fab, F(ab′)₂, and single domain VHH fragments such as those of camelid origin.

Single-chain antibody fragments, also known as single-chain antibodies (scFvs), are recombinant polypeptides which typically bind antigens or receptors; these fragments contain at least one fragment of an antibody variable heavy-chain amino acid sequence (VH) tethered to at least one fragment of an antibody variable light-chain sequence (VL) with or without one or more interconnecting linkers. Such a linker may be a short, flexible peptide selected to assure that the proper three-dimensional folding of the VL and VH domains occurs once they are linked so as to maintain the target molecule binding-specificity of the whole antibody from which the single-chain antibody fragment is derived. Generally, the carboxyl terminus of the VL or VH sequence is covalently linked by such a peptide linker to the amino acid terminus of a complementary VL and VH sequence. Single-chain antibody fragments can be generated by molecular cloning, antibody phage display library or similar techniques. These proteins can be produced either in eukaryotic cells or prokaryotic cells, including bacteria.

Single-chain antibody fragments contain amino acid sequences having at least one of the variable regions or CDRs of the whole antibodies described in this specification, but are lacking some or all of the constant domains of those antibodies. These constant domains are not necessary for antigen binding, but constitute a major portion of the structure of whole antibodies. Single-chain antibody fragments may therefore overcome some of the problems associated with the use of antibodies containing part or all of a constant domain. For example, single-chain antibody fragments tend to be free of undesired interactions between biological molecules and the heavy-chain constant region, or other unwanted biological activity. Additionally, single-chain antibody fragments are considerably smaller than whole antibodies and may therefore have greater capillary permeability than whole antibodies, allowing single-chain antibody fragments to localize and bind to target antigen-binding sites more efficiently. Also, antibody fragments can be produced on a relatively large scale in prokaryotic cells, thus facilitating their production. Furthermore, the relatively small size of single-chain antibody fragments makes them less likely than whole antibodies to provoke an immune response in a recipient.

Fragments of antibodies that have the same or comparable binding characteristics to those of the whole antibody may also be present. Such fragments may contain one or both Fab fragments or the F(ab′)₂ fragment. The antibody fragments may contain all six CDRs of the whole antibody, although fragments containing fewer than all of such regions, such as three, four or five CDRs, are also functional.

### Protein Production

The antibody-cytokine trap proteins are generally produced recombinantly, using mammalian cells containing a nucleic acid engineered to express the protein. Although one example of a suitable cell line and protein production method is described in Examples 1 and 2, a wide variety of suitable vectors, cell lines and protein production methods have been used to produce antibody-based biopharmaceuticals and could be used in the synthesis of these antibody-cytokine trap proteins.

### Therapeutic Indications

This invention relates to a combination therapy for the treatment of cancer, or reduction in tumor growth, particularly to a combination of (i) a bifunctional molecule comprising a TGFβRII or fragment thereof capable of binding TGFβ and an antibody, or antigen binding fragment thereof, that binds to an immune checkpoint protein, such as Programmed Death Ligand 1 (PD-L1) and (ii) at least one additional anti-cancer therapeutic agent. The anti-cancer therapeutic agents include, for example, radiation, chemotherapeutic agents, biologics, or vaccines. In certain embodiments of the invention, the combination therapy provides for a synergistic anti-cancer effect.

Exemplary cancers include colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, non-small cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodysplastic syndromes.

The cancer or tumor to be treated with an anti-PD-L1/TGFβ Trap, in combination with one or more additional anti-cancer therapeutic reagents, such as chemotherapy and/or radiation therapy, may be selected based on the expression or elevated expression of PD-L1 and TGFβ in the tumor, the correlation of their expression levels with prognosis or disease progression, and preclinical and clinical experience on the sensitivity of the tumor to treatments targeting PD-L1 and TGFβ. Such cancers or tumors include but are not limited to colorectal, breast, ovarian, pancreatic, gastric, prostate, renal, cervical, bladder, head and neck, liver, non-small cell lung cancer, melanoma, Merkel cell carcinoma, and mesothelioma.

### Pharmaceutical Compositions

The present invention also features pharmaceutical compositions that contain a therapeutically effective amount of a protein described herein for use in the therapeutic methods of the invention. The composition can be formulated for use in a variety of drug delivery systems. One or more physiologically acceptable excipients or carriers can also be included in the composition for proper formulation. Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

The pharmaceutical compositions are intended for parenteral, intranasal, topical, oral, or local administration, such as by a transdermal means, for therapeutic treatment. The pharmaceutical compositions can be administered parenterally (e.g., by intravenous, intramuscular, or subcutaneous injection), or by oral ingestion, or by topical application or intraarticular injection at areas affected by the vascular or cancer condition. Additional routes of administration include intravascular, intra-arterial, intratumor, intraperitoneal, intraventricular, intraepidural, as well as nasal, ophthalmic, intrascleral, intraorbital, rectal, topical, or aerosol inhalation administration. Thus, the invention provides compositions for parenteral administration that comprise the above mention agents dissolved or suspended in an acceptable carrier, preferably an aqueous carrier, e.g., water, buffered water, saline, PBS, and the like. The compositions may contain pharmaceutically acceptable auxiliary substances as required to approximate physiological conditions, such as pH adjusting and buffering agents, tonicity adjusting agents, wetting agents, detergents and the like. The invention also provides compositions for oral delivery, which may contain inert ingredients such as binders or fillers for the formulation of a tablet, a capsule, and the like. Furthermore, this invention provides compositions for local administration, which may contain inert ingredients such as solvents or emulsifiers for the formulation of a cream, an ointment, and the like.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

### Treatments

Determining the dosage and duration of treatment according to any aspect of the present invention is well within the skills of a professional in the art. The skilled artisan is readily able to monitor patients to determine whether treatment should be started, continued, discontinued or resumed. The amount of the antibody-TGFβ trap, the anti-cancer therapeutic, or dosage of radiation, for carrying out the combination treatment methods of the invention will vary depending on factors such as the condition being treated, the overall health of the patient, and the method, route and dose of administration.

According to certain embodiments the antibody-TGFβ trap, and the at least one additional anti-cancer agent, is administered at a therapeutic amount known to be used for treating the specific type of cancer. According to other embodiments, due to the observed synergistic effects associated with the combination therapy of the invention, the antibody-TGFβ trap, and the at least one additional anti-cancer agent can be administered in an amount that is lower than the therapeutic amount known to be used in monotherapies for treating the cancer.

The optimal dose of the antibody-TGFβ trap is based on the percent receptor occupancy by the antibody moiety to achieve maximal therapeutic effect because the cytokine trap is used in a large excess. For example, the therapeutic dose for a monoclonal antibody targeting a cellular receptor is determined such that the trough level is around 10 to 100 μg/ml, i.e., 60 to 600 nM (for antibody with a dissociation constant (K_{D}) of 6 nM, this trough level would ensure that between 90 to 99% of the target receptors on the cells are occupied by the antibody). This is in large excess of cytokines, which are typically present in pg to ng/ml in circulation.

The optimal dose of antibody-TGFβ trap polypeptide for use in the therapeutic methods of the invention will depend on the disease being treated, the severity of the disease, and the existence of side effects. The optimal dose can be determined by routine experimentation. For parenteral administration a dose between 0.1 mg/kg and 100 mg/kg, alternatively between 0.5 mg/kg and 50 mg/kg, alternatively, between 1 mg/kg and 25 mg/kg, alternatively, between 10 mg/kg and 25 mg/kg, alternatively, between 5 mg/kg and 20 mg/kg, alternatively between 2 mg/kg and 10 mg/kg, alternatively, between 5 mg/kg and 10 mg/kg, is administered and may be given, for example, once weekly, once every other week, once every third week, or once monthly per treatment cycle. In some embodiments of the invention, the effective dose of the antibody-TGFβ trap required to achieve a therapeutic effect in combination therapies will be less than that required in an antibody-TGFβ trap monotherapy to achieve a similar therapeutic effect.

In some embodiments of the invention, the effective dose will be about 2-10 times less than that required in an antibody-TGFβ trap monotherapy to achieve a similar therapeutic effect. In another embodiment, the effective dose will be about 2-5 times less than that required in an antibody-TGFβ trap monotherapy to achieve a similar therapeutic effect.

The effective dosage of the additional chemotherapeutic reagent, or radiation therapy, for use in combination with an antibody-TGFβ trap for treatment of cancer may vary depending on the particular compound or pharmaceutical composition employed, the mode of administration, the condition being treated and the severity of the condition being treated. A physician or clinician of ordinary skill can readily determine the effective amount of each additional chemotherapeutic reagent, or radiation, necessary to treat or prevent the progression of the cancer. In some embodiments of the invention, the effective dose of the additional chemotherapeutic reagent or radiation therapy required to achieve a therapeutic effect in the combination therapy of the invention will be less than that required in chemotherapeutic or radiation monotherapies to achieve a similar therapeutic effect.

According to the methods of the invention, chemotherapeutic agents can be administered in combination with an antibody-cytokine trap molecule to treat cancer or reduce tumor growth. Such chemotherapeutic agents include, for example, alkylating agents, antimetabolites, anthracyclines, plant alkaloids, topoisomerase inhibitors, antineoplastic antibiotics, hormonal agents, anti-angiogenic agents, differentiation inducing agents, cell growth arrest inducing agents, apoptosis inducing agents, cytotoxic agents and other anti-tumor agents. Such drugs may affect cell division or DNA synthesis and function in some way. Representative chemotherapeutic agents include, but are not limited to alkylating agents (such as cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil (5-FU), gemcitabine, methotrexate, cytosine arabinoside, fludarabine, and floxuridine), antimitotics (including taxanes such as paclitaxel and decetaxel and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), and topoisomerase inhibitors (including camptothecins such as irinotecan and topotecan and derivatives of epipodophyllotoxins such as amsacrine, etoposide, etoposide phosphate, and teniposide).

In certain embodiments, platinum-based therapeutics such as cisplatin, carboplatin and oxaliplatin are utilized. Other anti-cancer agents whose treatment and effects can benefit from combination with anti-PD-L1/TGFβ Trap molecule include antimetabolites, such as flurouracil (5-FU), which interfere with DNA synthesis. In certain embodiments, combinations of one or more chemotherapeutic agents may be administered with the anti-PD-L1/TGFβ Trap molecule. In other embodiments, combinations of one or more chemotherapeutic agents may be administered with and radiation therapy and the anti-PD-L1/TGFβ Trap molecule.

In a specific embodiment of the invention, oxaliplatin may be administered in a dose of between 20 mg/m² and 200 mg/m², alternatively between 40 mg/m² and 160 mg/m², alternatively, between 60 mg/m² and 145 mg/m², alternatively, between 85 mg/m² and 135 mg/m², alternatively between 40 mg/m² and 65 mg/m².

In a specific embodiment of the invention, 5-FU may be administered in a dose of between 100 mg/m² and 3000 mg/m², alternatively, between 250 mg/m² and 2400 mg/m², alternatively, between 400 mg/m² and 1500 mg/m², alternatively, between 200 mg/m² and 600 mg/m². In an embodiment of the invention, the 5-FU dose may be administered, for example, by infusion over an extended period of time.

In a specific embodiment of the invention, leucovorin may also be administered, to enhance the effects of the 5-FU or to decrease the side effects associated with chemotherapy.

In a specific embodiment of the invention, the following chemotherapeutic regimen is provide as an example for use in combination with the anti-PD-L1/TGFβ Trap molecule. On day 1, 85 mg/m² of oxaliplatin and 200 mg/m² of leucovorin are administered followed 2 hours later by administration of 400 mg/m² bolus of 5-FU and 600 mg/m² infusion of 5-FU. On day 2, 200 mg/m² of leucovorin is administered followed 2 hours later by 400 mg/m² bolus of 5-FU and 600 mg/m² infusion of 5-FU.

In another embodiment of the invention, the chemotherapeutic regimen includes, for example, administration on day 1 of a 85 mg/m² dose of oxaliplatin, a 400 mg/m² dose of leucovorin, a 400 mg/m² IV bolus dose of 5-FU and a 600 mg/m² infusion of 5-FU followed by 1200 mg/m²/day×2 days (total 2400 mg/ml² over 46-48 hours) IV continuous infusion. The treatment is repeated every 2 weeks.

In another embodiment, a 2 hour infusion of 400 mg/m² of leucovorin is administered followed by a 5-FU 46-hour infusion of 2400 mg/m². Oxaliplatin is also infused for two hours on day 1 at a dose of 130 mg/m². The treatment is repeated every two weeks.

According to the methods of the present invention, radiation can be administered in combination with an antibody-cytokine trap molecule to treat cancer. Radiation therapy typically uses a beam of high-energy particles or waves, such as X-rays and gamma rays, to eradicate cancer cells by inducing mutations in cellular DNA. Cancer cells divide more rapidly than normal cells, making tumor tissue more susceptible to radiation than normal tissue. Any type of radiation can be administered to a patient, so long as the dose of radiation is tolerated by the patient without significant negative side effects. Suitable types of radiotherapy include, for example, ionizing radiation (e.g., X-rays, gamma rays, or high linear energy radiation). Ionizing radiation is defined as radiation comprising particles or photons that have sufficient energy to produce ionization, i.e., gain or loss of electrons. The effects of radiation can be at least partially controlled by the clinician. The dose of radiation is preferably fractionated for maximal target cell exposure and reduced toxicity. Radiation can be administered concurrently with radiosensitizers that enhance the killing of tumor cells, or with radioprotectors (e.g., IL-1 or IL-6) that protect healthy tissue from the harmful effects of radiation. Similarly, the application of heat, i.e., hyperthermia, or chemotherapy can sensitize tissue to radiation.

The source of radiation can be external or internal to the patient. External radiation therapy is most common and typically involves directing a beam of high-energy radiation (a particle beam) to a tumor site through the skin using, for instance, a linear accelerator. While the beam of radiation is localized to the tumor site, it is nearly impossible to avoid exposure of normal, healthy tissue. However, external radiation is usually well tolerated by patients.

In another example, radiation is supplied externally to a patient using gamma rays. Gamma rays are produced by the breakdown of radioisotopes such as cobalt 60. Using a treatment approach called Stereotactic Body Radiation Therapy (SBRT), gamma rays can be tightly focused to target tumor tissue only, such that very little healthy tissue is damaged. SBRT can be used for patients with localized tumors. On the other hand, X-rays, produced by a particle accelerator, can be used to administer radiation over a larger area of the body.

Internal radiation therapy involves implanting a radiation-emitting source, such as beads, wires, pellets, capsules, etc., inside the body at or near the tumor site. The radiation used comes from radioisotopes such as, but not limited to, iodine, strontium, phosphorus, palladium, cesium, iridium, phosphate or cobalt. Such implants can be removed following treatment, or left in the body inactive. Types of internal radiation therapy include, but are not limited to, brachytherapy, interstitial irradiation, and intracavity irradiation. A currently less common form of internal radiation therapy involves biological carriers of radioisotopes, such as with radioimmunotherapy wherein tumor-specific antibodies bound to radioactive material are administered to a patient. The antibodies bind tumor antigens, thereby effectively administering a dose of radiation to the relevant tissue.

Radiation therapy is useful as a component of a regimen to control the growth of a primary tumor (see, e.g. Comphausen et al. (2001) “Radiation Therapy to a Primary Tumor Accelerates Metastatic Growth in Mice,” Cancer Res. 61:2207-2211). Although radiation therapy alone may be less effective at treating cancer, combining radiation with an anti-PD-L1/TGFβ Trap molecule as described herein, can enhance the local and systemic efficacy of radiation therapy.

Because radiation kills immune effector cells, the dose and timing of the radiation is important. T cells and dendritic cells in an irradiated tumor decrease immediately after irradiation; however, T-cell levels rebound higher than baseline levels. No matter the method of administration, a complete daily dose of radiation can be administered over the course of one day. Preferably, the total dose is fractionated and administered over several days. Accordingly, a daily dose of radiation will comprise approximately 1-50 Gy/day, for example, at least 1, at least 2, at least 3, 1-4, 1-10, 1-20, 1-50, 2-4, 2-10, 2-20, 2-25, 2-50, 3-4, 3-10, 3-20, 3-25, 3-50 Gy/day.

The daily dose can be administered as a single dose, or can be a “microfractionated” dose administered in two or more portions over the course of a day. When internal sources of radiation are employed, e.g., brachytherapy or radio-immunotherapy, the exposure time typically will increase, with a corresponding decrease in the intensity of radiation.

According to some embodiments of the invention, the antibody-TGFβ trap and the at least one additional anti-cancer agent are administered simultaneously. According to another embodiment, the antibody-TGFβ trap and the at least one additional anti-cancer agent are administered sequentially.

The dosing frequency of the antibody-TGFβ trap and the at least one additional anti-cancer therapeutic agent may be adjusted over the course of the treatment, based on the judgment of the administering physician.

### EXAMPLES

The invention now being generally described, will be more readily understood by reference to the following examples, which are included merely for purposes of illustration of certain aspects and embodiments of the present invention, and are not intended to limit the scope of the invention in any way.

### Example 1—DNA Construction and Protein Expression

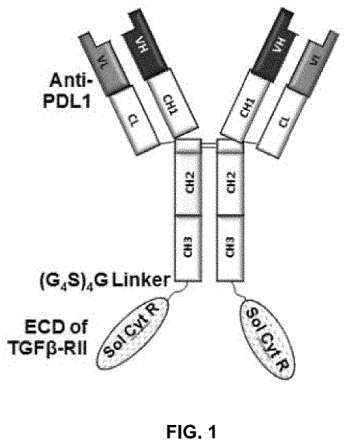

Anti-PD-L1/TGFβ Trap is an anti-PD-L1 antibody-TGFβ Receptor II fusion protein. The light chain of the molecule is identical to the light chain of the anti-PD-L1 antibody (SEQ ID NO:1). The heavy chain of the molecule (SEQ ID NO:3) is a fusion protein comprising the heavy chain of the anti-PD-L1 antibody (SEQ ID NO:2) genetically fused to via a flexible (Gly₄Ser)₄Gly linker (SEQ ID NO:11) to the N-terminus of the soluble TGFβ Receptor II (SEQ ID NO:10). At the fusion junction, the C-terminal lysine residue of the antibody heavy chain was mutated to alanine to reduce proteolytic cleavage. For expression of anti-PD-L1/TGFβ Trap, the DNA encoding the anti-PD-L1 light chain (SEQ ID NO:4) and the DNA encoding the anti-PD-L1/TGFβ Receptor II (SEQ ID NO:5) in either the same expression vector or separate expression vectors were used to transfect mammalian cells using standard protocols for transient or stable transfection. Conditioned culture media were harvested and the anti-PD-L1/TGFβ Trap fusion protein was purified by standard Protein A Sepharose chromatography. The purified protein comprising one anti-PD-L1 antibody and two soluble TGFβ Receptor II molecules (FIG. 1) has an estimated molecular weight (MW) of about 190 kilodaltons on size exclusion chromatography and SDS-polyacrylamide electrophoresis under non-reducing conditions. Under reducing conditions, the light and heavy chains have apparent MW of 28 and 75 kilodaltons, respectively.

The anti-PD-L1(mut)/TGFβ Trap fusion protein, which contains an analogous heavy chain fusion polypeptide (SEQ ID NO:7) and a light chain with the mutations A31G, D52E, R99Y in the variable region that abrogate the binding to PD-L1 (SEQ ID NO:6), was similarly prepared. It was used in subsequent experiments as a TGFβ Trap control.

### Example 2—Production of Anti-PD-L1/TGFβ Trap as a Biotherapeutic

The anti-PD-L1/TGFβ Trap produced by transient transfection of human embryonic kidney 293 (HEK) cells was found to contain varying degrees of a clipped species, which appeared as a faint band with an apparent MW of about 60 kD on SDS-PAGE under reducing conditions. This band was confirmed to be the heavy chain of the anti-PD-L1/TGFβ Trap cleaved at a site in the N-terminal portion of TGFβRII close to the fusion junction.

Stable clones expressing anti-PD-L1/TGFβ Trap were generated in the CHO-S host cell line, which was pre-adapted for growth in serum-free media in suspension culture. Cells were transfected with an expression vector containing a gene encoding the anti-PD-L1-TGFβRII protein and a glutamine synthetase selection marker. Subsequent selection of stable integrants was made with L-methionine sulfoximine (MSX). Anti-PD-L1/TGFβ Trap expressing cell lines were generated using a minipool approach, followed by the deposition of single cells into 384-well plates, using a Beckton-Dickinson fluorescence activated cell sorter (FACS Aria II). Growth, productivity, and protein quality were evaluated in a generic platform fed-batch assay. Based on these analyses, 14 clones were selected as lead candidates for further studies. A stability study with the clones was carried out to ˜90 PDL (population doubling level) from research cell banks established during scale up of clones. At the conclusion of mini-pool development it was discovered that the heavy chain-linker-TGFβRII subunit underwent clipping, as was seen in transient expression. All clones in the stability study produced the clipped species, although it was shown in the protein A-purified material that the percent clipped species relative to the intact subunit varied with each clone. In addition, an improved purification process consisting a protein A chromatography followed by strong cation exchange was developed to reduce co-purification of the clipped species. Even with the improved process, purified material with the required final levels of clipped species of <5% could only be achieved using clones producing low levels of clipping. Based on these combined analyses, clone 02B15 was selected as the final candidate clone. Analysis of anti-PD-L1/TGFβ Trap expressed by this clone at zero PDL, thirty PDL, sixty PDL and ninety PDL shows that the percentage of clipping did not increase with population doubling levels.

### Example 3—Combination of Chemotherapy and Anti-PD-L1/TGFβ Trap in a Subcutaneous MC38 Tumor Mouse Model

Colorectal cancer (CRC) is the third most common cancer in males and the second in females, with over 1.2 million new cases worldwide. Despite significant progress in treatment over the last decade, CRC is the fourth most common cause of cancer-related deaths. Thus, novel treatment modalities are needed. In the working example set forth below, the efficacy of the anti-PD-L1/TGFβ Trap molecule was investigated in combination with oxaliplatin (Ox) and 5-fluorouracil (5-FU) based therapy in a murine model of colorectal cancer.

Combination treatment with anti-PD-L1/TGFβ Trap and the chemotherapeutic reagent Ox/5-FU in mice with subcutaneous MC38 tumors resulted in significant inhibition of tumor growth. These preclinical data support a strategy of combining chemotherapy (Ox/5-FU) with anti-PD-L1/TGFβ Trap immunotherapy for the treatment of colorectal cancer in the clinic.

### Materials and Methods

The MC38 tumor cell line was obtained from American Type Culture Collection (ATCC). The MC38 cell line was tested and verified to be free of adventitious viruses and mycoplasma. C57BL/6 mice, 8-12 weeks of age, were obtained from Charles River Laboratories. B6.129S2-Ighm^{tm1Cgn}/J mice, 8-12 weeks of age, were from Jackson Laboratories.

Test material doses were as follows: Anti-PD-L1/TGFβ Trap: 24.6 mg/kg; 492 μg/mouse; 2.46 mg/mL; 0.2 mL dose volume administered intravenously. Fluorouracil (5-FU): 60.0 mg/kg; 120 μg/mouse; 6.00 mg/mL; 0.02 mL dose volume administered intravenously. Oxaliplatin: 5.0 mg/kg; 10 μg/mouse 0.500 mg/mL 0.02 mL administered i.p. The value in mg/kg was approximate, assuming an average body weight of 20 g per mouse.

The negative control was an inactive isotype control (Anti-PD-L1(mut)) administered at a test concentration of 400 μg/mouse.

Cell Culture. MC38 cells were cultured under aseptic conditions in Dulbecco's minimal essential medium (DMEM) containing 10% heat-inactivated fetal bovine serum and maintained at 37° C. and 5% CO². Cells were passaged upon reaching 50-70% confluence at a ratio of 1:5, for a total of 2 passages prior to in vivo implantation. Cells were harvested by trypsinization and viable cell counts were determined using a hemocytometer and trypan blue exclusion staining. All cell culture reagents were purchased from Life Technologies (Gaithersburg, Md.).

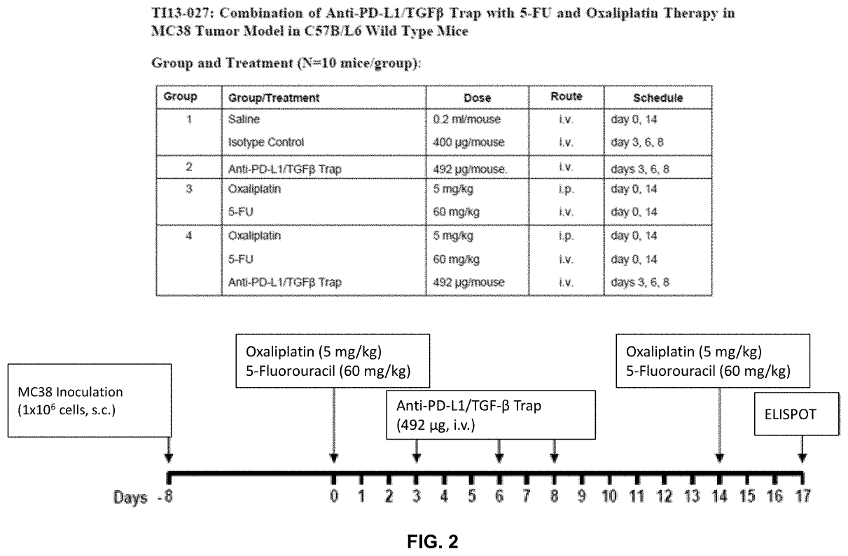

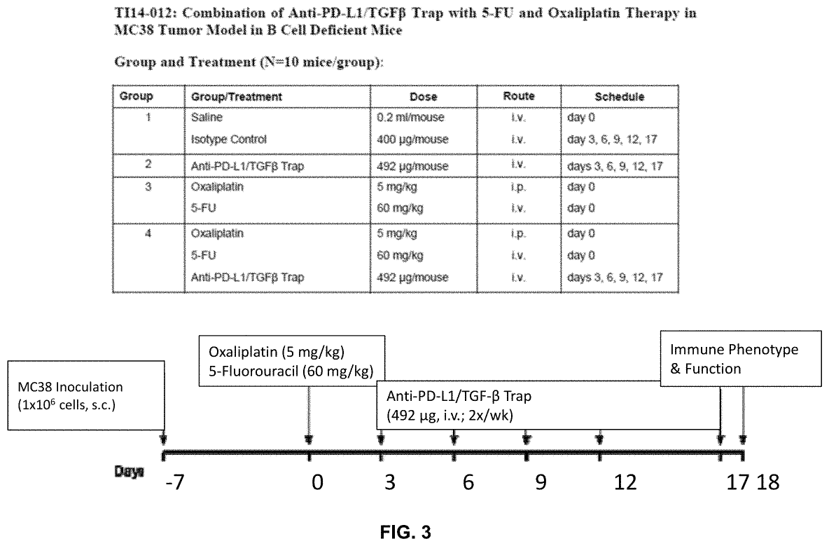

MC38 Tumor Model. In study TI13-027, MC38 tumor cells (1×10⁶ cells/mouse) were suspended in 100 μL of sterile PBS and implanted into right flank of C57BL/6 mice. When the tumor sizes reached an average of ˜45 mm³, the mice were randomized into 4 groups (N=10 mice/group) to initiate therapy. Treatments were administered as per the dose schedules as set forth in FIG. 2 and FIG. 3. The length (L), width (W) and height (H) of tumor was measured with digital caliper and recorded automatically to computer twice per week using WinWedge software. Body weights were also recorded twice per week to assess tolerability. Tumor volumes were calculated by the Ellipsoid volume formulas: Volume=π/6*(L×W×H); where L=length, W=width and H=height of the tumor. Efficacy was determined by measuring tumor volume throughout the duration of the in vivo study and the tumor weights were measured at study termination point as described below. All animals were sacrificed on day 17 and tumors were excised and weighed. The spleens were harvested for IFN-γ ELISPOT analyses.

In study TI14-012, MC38 tumor cells were injected into B6.129S2-Ighm^{tm1Cgn}/J mice as described above. All other procedures for evaluation of tumor growth and treatment efficacy were also as described above.

IFN-γ ELISpot Assay. The enzyme-linked immunosorbent spot (ELISpot) assay was used to measure the cytotoxic T lymphocytes (CTL) response against the p15E antigen, which is a known T cell rejection epitope in MC38 tumors (Yang and Perry-Lalley J Immunotherapy 2000; 23:177-183). The ELISpot assay measures the frequency of IFN-γ producing CD8⁺ T cells following co-culture with antigen presenting cells (APC) loaded with the p15E epitope KPSWFTTL (SEQ ID NO:49). APCs loaded with an irrelevant peptide, SIINFEKL (SEQ ID NO:50), derived from chicken ovalbumin (OVA) served as a negative control. Positive control samples were stimulated with PMA and ionomycin, which triggers a non-specific activation of cytotoxic T lymphocytes. ELISpot assay was performed using a mouse IFN-γ ELISpot Kit from BD Biosciences according to the manufacturer's instructions. On day 17 of study TI13-027, the spleens of N=5 mice/group were harvested, processed into single cell suspensions, stimulated with P15E peptide at a final concentration of 1 μg/mL, and then cultured at 37° C. for 7 days. After the in vitro stimulation, CD8⁺ T cells were isolated by magnet activated cell sorting using the CD8⁺ T cell isolation kit (Miltenyi Biotech) and the AutoMACS Pro Separator. To establish the co-culture system for the in vitro stimulation ELISpot assay, APCs derived from naive mouse splenocytes were pulsed with the KPSWFTTL (SEQ ID NO:49) peptide or the irrelevant SIINFEKL (SEQ ID NO:50) peptide for one hour and then irradiated with 2 Gy in the GammaCell 40 Exactor. Isolated CD8⁺ T cells (1×10⁵ cells/well) from experimental mice were cultured in triplicate in ELISpot assay plates (anti-IFN-γ antibody coated) with peptide-pulsed and irradiated APCs (2.5×10⁵ cells/well).

On day 18 of study TI14-012, an ex vivo ELISpot assay was established in which the spleens of N=5 mice/group were harvested, processed into single cell suspensions, and CD8⁺ T cells were isolated with magnet activated cell sorting using the CD8⁺ T cell isolation kit (Miltenyi Biotech) and the AutoMACS Pro Separator. To establish the co-culture system for the ex vivo ELISpot assay, APCs derived from naive mouse splenocytes were pulsed with the KPSWFTTL (SEQ ID NO:49) peptide or the irrelevant SIINFEKL (SEQ ID NO:50) peptide for one hour and then irradiated with 2 Gy in the GammaCell 40 Exactor. Isolated CD8⁺ T cells (5×10⁵ cells/well) from experimental mice were cultured in triplicate in ELISpot assay plates (anti-IFN-γ antibody coated) with peptide-pulsed and irradiated APCs (5×10⁵ cells/well).

In both experiments, the experimental CD8+ T cells were co-cultured with peptide-pulsed APCs at 37° C. for 19-20 hours prior to being removed from the assay plate. A biotinlyated anti-IFN-γ antibody was added to each well of the plate, followed by a wash step, and then addition of a streptavidin-HRP detection conjugate. After another wash step, the plate was incubated with a chromogenic substrate solution; the reaction was monitored and then stopped by rinsing the plate with water. The number of IFN-γ positive spots in each well of the assay plate was counted using a CTL-Immunospot S5UV Analyzer (Cellular Technology Limited). The data are represented as the mean number of spots/well±SEM.

Mortality checks were performed once daily during the study. Clinical Observations. Clinical signs (such as ill health and behavioral changes) were recorded for all animals once daily during the study using the body condition (BC) scoring system as previously described (Ullman-Cullere and Foltz, Lab Anim Sci. 1999; 49:319-23). Moribund mice were humanely euthanized by CO² asphyxiation. Body weights for all animals on the study were recorded twice per week including the termination day of each group. Tumors volumes were measured in three dimensions with digital calipers and recorded automatically to a computer twice per week using WinWedge software for the duration of the experiment. Tumor volumes were calculated using the ellipsoid volume formula: Volume=0.5236 (L×W×H); where L=length, W=width and H=height of the tumor. At the time of sacrifice, the primary tumor was excised and weighed as a secondary efficacy endpoint. The frequency of IFN-γ producing, P15E-specific CD8⁺ T cells was quantified by ELISpot assay. Mouse IFN-γ ELISpot assays were performed using a mouse IFN-γ ELISpot kit (BD Biosciences) according to the manufacturer's instructions.

Statistical Analysis. Tumor volumes were measured twice per week throughout the study period. Tumor volume data was presented as the mean±standard error of the mean (SEM). The tumor growth inhibition % T/C ratio was calculated as the tumor volume of the treatment group divided by the tumor volume of control group and then multiplied 100. Tumor volume data was log transformed and two-way, repeated measures ANOVA with Tukey's correction for multiple comparisons was performed to measure statistical differences between treatment groups. T/C was calculated as the tumor volume of the treatment group divided by the tumor volume of control group. Tumor weights were measured at study completion. The data was represented as the mean±SEM. The % T/C ratio was calculated as the tumor weight of the treatment group divided by the tumor weight of control group and then multiplied 100. Tumor weight data was evaluated with one-way ANOVA with Tukey's correction for multiple comparisons to measure statistical differences between treatment groups. IFN-γ ELISpot data was expressed as the mean±SEM. A One-Way ANOVA with Tukey's correction for multiple comparisons was used for statistical analyses using GraphPad Prism Software. p<0.05 was determined to be statistically significant.

### Results

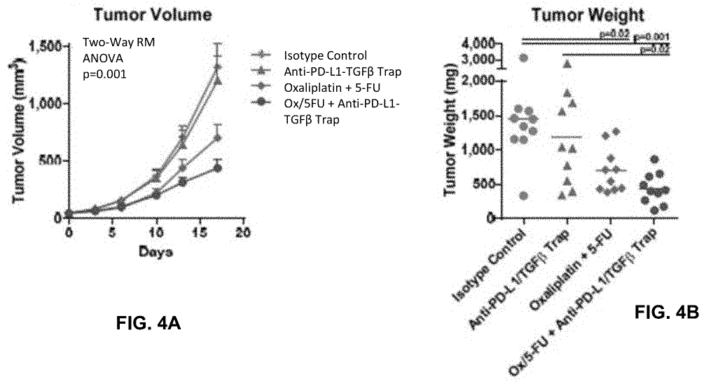

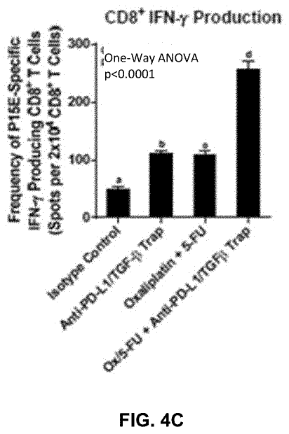

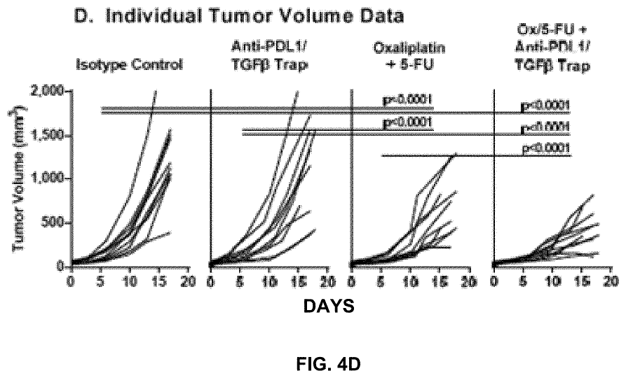

Due to the immunogenicity caused by the fully humanized antibody in B cell competent mice, the anti-PD-L1/TGFβ Trap molecule could only be administered three times within one week in the C57BL/6 wild-type mice in study TI13-027. Consequently, significant antitumor activity was not observed with anti-PD-L1/TGFβ Trap monotherapy (% T/C=91% in tumor volume; see FIG. 4). The Oxaliplatin/5-FU treatment induced significant tumor growth inhibition in the MC38 subcutaneous tumor model compared to the Isotype control (% T/C=53.2% in tumor volume; p<0.0001). Combination therapy with anti-PD-L1/TGFβ Trap and oxaliplatin/5-FU significantly inhibited MC38 tumor growth compared with the control group (% T/C=33.2% in tumor volume; p<0.0001). Moreover, the combination of anti-PD-L1/TGFβ Trap and oxaliplatin/5-FU significantly improved tumor growth control relative to oxaliplatin/5-FU alone (439.6 mm³ vs. 703.7 mm³ in tumor volume; p<0.0001). The same trend was observed in which the combination treatment produced statistically significant improvements in tumor growth control compared to anti-PD-L1/TGFβ Trap alone (439.6 mm³ vs. 1204.0 mm³; p<0.0001) (see FIG. 4A-D and Table 1).

TABLE 1
Results of Tumor Volume, Tumor Weight, and ELISpot Assay
at Study Completion (C57BL/6 mice; Study TI13-027)

| | | | % T/C of | | % T/C of | |
|---|---|---|---|---|---|---|
| | | Tumor volume | tumor | Tumor weight | tumor | IFN-γ |
| Group | Treatment | (mm³) [symbol] | volume | (mg) [symbol] | weight | ELISPOT |
| G1 | Isotype Control | 1323.5 ± 199.1 | 100 | 1448.5 ± 220.0 | 100 | 49.0 ± 4.0 |
| | (Anti-PD-L1(mut) | | | | | |
| G2 | Anti-PD-L1/TGFβ Trap | 1204.0 ± 217.2 | 91 | 1196.8 ± 248.8 | 82.6 | 113.3 ± 4.5 |
| G3 | Qxaliplatin + 5-FU | 703.7 ± 115.6 | 53.2 | 701.4 ± 102.8 | 48.4 | 108.3 ± 23.6 |
| G4 | Oxaliplatin/5-FU + | 439.6 ± 71.1 | 33.2 | 438.2 ± 71.9 | 30.3 | 258.0 ± 14.3 |
| | Anti-PD-L1/TGFβ Trap | | | | | |

[symbol]

indicates data missing or illegible when filed

Finally, anti-PD-L1/TGFβ Trap monotherapy or the oxaliplatin/5-FUmonotherapy were observed to significantly increase the frequency of IFN-γ producing CD8⁺ T cells compared to the Isotype control group as measured by ELISpot assay (p<0.05 and p<0.05, respectively). The combination of anti-PD-L1/TGFβ Trap and oxaliplatin/5-FU significantly enhanced the frequency of P15E-specific, IFN-γ producing CD8+ T cells relative to either monotherapy group (p<0.05; see FIG. 4C).

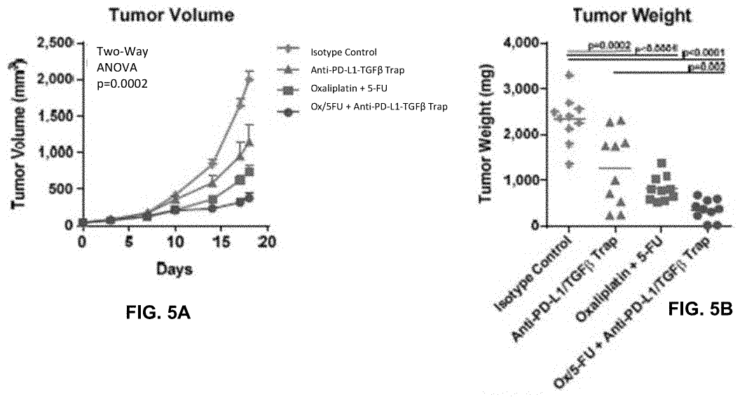

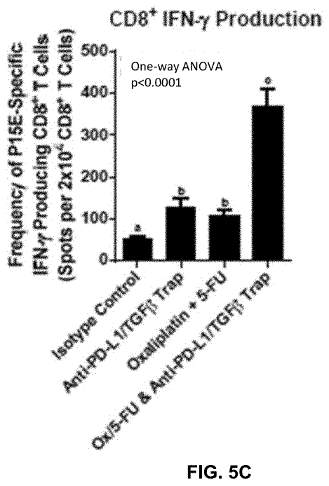

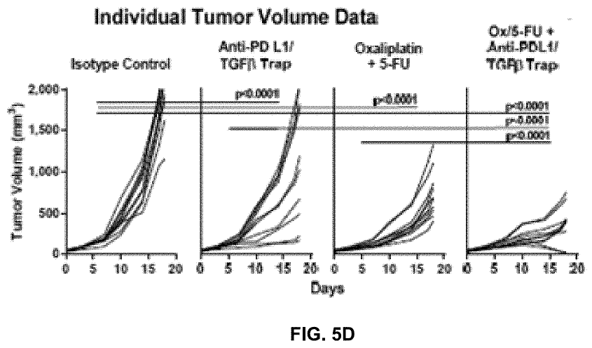

In study TI14-012, utilizing B cell deficient mice to avoid the mouse antibody against human antibody (MAHA) response, experimental animals were treated five times with anti-PDL1/TGFβ Trap. Not surprisingly, greater antitumor activity was observed with anti-PDL1/TGFβ Trap monotherapy (% T/C=57.3% in tumor volume) compared to study TI13-027 in which wild-type mice were treated only three times (% T/C=91% in tumor volume). In study TI14-012, the combined treatment with anti-PD-L1/TGFβ Trap and oxaliplatin/5-FU was significantly more effective compared to either of the monotherapies (p<0.0001) or the isotype control (p<0.0001) (see FIG. 5D and Table 2).

TABLE 2
Results of Tumor Volume, Tumor Weight, and ELISpot Assay at Study
Completion (B6.129S2-Ighm [symbol] /J mice; Study TI14-012)

| | | | % T/C of | | % T/C of | |
|---|---|---|---|---|---|---|
| | | Tumor volume | tumer | Tumor weight | tumor | IFN-γ |
| Group | Treatment | (mm³) [symbol] | volume | (mg) [symbol] | weight | ELISPOT |
| G1 | Isotype Control | 2003.4 ± 122.4 | 100 | 2336.2 ± 164.8 | 100 | 51.7 ± 5.5 |
| | (Anti-PD-L1(mut) | | | | | |
| G2 | Anti-PD-L1/TGFβ Trap | 1147.7 ± 234.9 | 57.3 | 1265.0 ± 256.5 | 54.1 | 160.3 ± 18.5 |
| G3 | Oxaliplatin + 5-FU | 743.9 ± 92.4 | 37.1 | 822.7 ± 86.0 | 35.2 | 107.7 ± 13.0 |
| G4 | Oxaliplatin/5-FU + | 380.8 ± 74.6 | 19.0 | 362.8 ± 70.9 | 15.5 | 369.7 ± 39.7 |
| | Anti-PD-L1/TGFβ Trap | | | | | |

[symbol]

indicates data missing or illegible when filed

Similarly, anti-PD-L1/TGFβ Trap monotherapy resulted in significantly increased frequencies of IFN-γ producing CD8⁺ T cells compared to the isotype control (see FIG. 5C; p<0.05). The combined treatment of anti-PD-L1/TGFβ Trap and oxaliplatin/5-FU resulted in a synergistic increase in the frequency of P15-specific, IFN-γ producing CD8⁺ T cells compared to either monotherapy group or the Isotype control (see FIG. 5C; p<0.05).

Anti-PD-L1/TGFβ Trap is a bifunctional antibody-cytokine receptor fusion protein designed to reverse both the cell-intrinsic and extrinsic immune suppression in the tumor microenvironment through dual targeting of the PD-1/PD-L1 axis and TGFβ signaling. In the studies described herein, significant MC38 tumor growth inhibition and the synergistic induction of P15E-specific CD8⁺ T cell IFN-γ production were observed with the combination of anti-PDL1/TGFβ Trap and Ox/5-FU treatment in mice with subcutaneous MC38 tumors. These effects on antitumor efficacy and immune response observed in wild-type mice were accentuated in B cell deficient mice. The difference is believed to be primarily due to administration of a greater number of doses of anti-PD-L1/TGFβ Trap and absence of the MAHA (mouse against human antibody) response in the B cell deficient mice. Taken together, these data demonstrate that components of chemotherapy (Ox/5-FU) can be effectively combined with anti-PDL1/TGFβ Trap therapy to enhance tumor growth inhibition and tumor-reactive CD8⁺ T cell responses in a mouse colorectal cancer model. In conclusion, the preclinical results support a combination for the treatment of colorectal cancer in the clinic.

### Example 4—Combination of Radiation Therapy and Anti-PD-L1/TGFβ Trap in a Intramuscular MC38 Tumor Mouse Model

The anti-PD-L1/TGFβ Trap molecule is comprised of the extracellular domain of the human TGFβRII (TGFβ Trap) covalently linked to the C-terminus of the heavy chain of a human anti-PD-L1 antibody. Anti-PD-L1/TGFβ Trap monotherapy has shown superior antitumor efficacy in multiple preclinical models. In the studies reported here, we investigated the antitumor activity of the anti-PD-L1/TGFβ Trap in combination with fractionated local radiation therapy in B6.12982-Ighm^{tm1Cgn}/J mice bearing intramuscular MC38 colorectal tumors. The data showed that the combination of radiation given as four fractionated doses of local radiation (360 rads/dose) and a single administration of anti-PD-L1/TGFβ Trap (55 μg) had remarkably synergistic antitumor effects resulting in tumor remission in 100% of the mice. In addition, the combination of radiation given as four fractionated doses of local radiation (500 rads/dose) and a single administration of the anti-PD-L1/TGFβ Trap (164 μg) elicited anti-cancer effects on tumors at sites distal to the tumor being irradiated, a demonstration of the abscopal effect, and an indication that such treatment would be useful in treating metastasis. By comparison, monotherapy with either radiation or anti-PD-L1/TGFβ Trap treatment alone resulted in a modest reduction in tumor burden. Furthermore, significant increases in the frequency of P15E-specific, IFN-γ producing CD8⁺ T cells were observed in the mice receiving the combination therapy. Finally, the combination therapy was associated with improved infiltration of MC38 tumors by effector CD8⁺ T cells and NK cells. These results indicate that anti-PD-L1/TGFβ Trap treatment synergizes with radiation to facilitate T cell mediated antitumor responses. The results described below support this combination strategy for potential clinical applications.

### Materials and Methods

Cell line: MC38 murine colon carcinoma cell line was a gift from the Scripps Research Institute. The cell line was tested and verified to be murine virus and mycoplasma free. Animals B6.129S2-Ighm^{tm1Cgn}/J mice (C57BL/6), 8-12 weeks of age, were obtained from Jackson Laboratories.

Test material doses were as follows: Anti-PD-L1/TGFβ Trap: 2.75 mg/kg; 55 μg/mouse; 13.75 mg/mL; 0.2 mL dose volume administered intravenously and anti-PD-L1/TGFβ Trap: 8.25 mg/kg; 164 μg/mouse; 41.25 mg/mL; 0.2 mL dose volume administered intravenously.

Negative controls was as follows: inactive isotype control (anti-PD-L1(mut) A11-121-6) was administered at a test concentration of either 133 μg/mouse or 45 μg/mouse.

MC38 cells were cultured under aseptic conditions in Dulbecco's minimal essential medium, containing 10% heat-inactivated fetal bovine serum, and maintained at 37° C. and 5% CO2. Cells were passaged upon reaching 50-70% confluence at a ratio of 1:5, for a total of 2 passages prior to in vivo implantation. Cells were harvested by trypsinization and viable cell counts were determined using a hemocytometer and trypan blue exclusion staining.

C38 tumor model: C57BL/6.12952-Ighm^{tm1Cgn}/J mice were implanted intramuscularly into the right thigh with 0.5×10⁶ viable MC38 tumor cells in 0.1 ml PBS on day −8. When the tumors had reached a mean volume of ˜128 mm³, mice were randomized into treatment groups. Treatment started on day 0 (8 days after tumor cell inoculation).

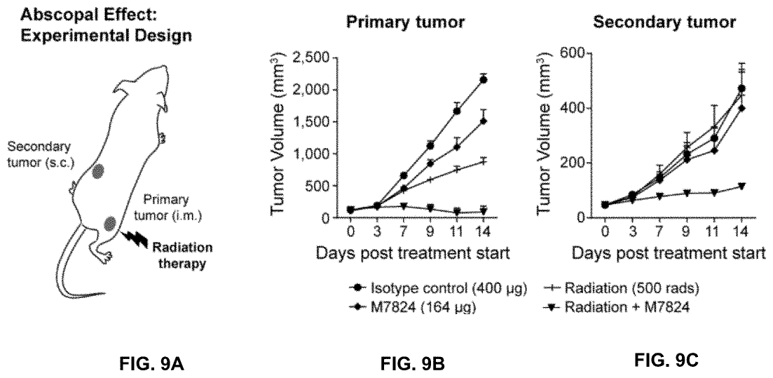

Localized radiation therapy can elicit anti-cancer effects at distal sites, a phenomenon known as an “abscopal” effect. To test the effect of the Anti-PD-L1/TGFβ Trap on the abscopal effect of radiation therapy, 7 days prior to treatment, mice were inoculated with 0.5×10⁶ viable MC38 tumor cells to generate a primary, intramuscular MC38 tumor in the right thigh, and with 1×10⁶ MC38 cells subcutaneously in the left flank to generate a secondary, subcutaneous MC38 tumor (FIG. 9A). Treatment commenced on day 7.

Radiotherapy: Mice were positioned on a dedicated plexiglass tray, and the whole body was protected by lead shielding except for the area of the tumor to be irradiated. Radiotherapy was delivered to the tumor field through the use of GammaCell 40 Exactor.

Enzyme-linked Immunosorbent Spot (ELISpot) Assay: The ELISpot assay was used to measure the cytotoxic T lymphocyte (CTL) response against the p15E antigen, which is a known T cell rejection epitope expressed by MC38 tumors (refer to Yang and Perry-Lalley 2000). The ELISpot assay measures the frequency of IFN-γ producing CD8⁺ T cells following co-culture with antigen presenting cells (APCs) loaded with the p15E epitope KPSWFTTL (SEQ ID NO:49). A PCs loaded with an irrelevant peptide derived from chicken ovalbumin (SIINFEKL (SEQ ID NO:50)) served as a negative control. Positive control samples were stimulated with PMA and ionomycin, which triggers a non-specific activation of CTLs. The ELISpot assay was performed using a kit from BD Biosciences. On study day 14, the spleen was harvested from one mouse in each study group and processed into a single cell suspension. The CD8⁺ T cells were isolated by magnet activated cell sorting using the CD8⁺ T cell isolation kit from Miltenyi Biotech, and the AutoMACS Pro Separator. CD8⁺ T cells were then seeded in ELISpot assay plates (anti-IFN-γ antibody coated) in co-culture with APC derived from naive mouse splenocytes pulsed with the KPSWFTTL (SEQ ID NO:49) peptide for one hour, and then irradiated with 2 Gy in the GammaCell 40 Exactor. After incubation at 37° C. for 16-20 hours, the cells were removed from the assay plate. A biotinlyated anti-IFN-γ antibody was added to each well of the plate, followed by a wash step, and then addition of a streptavidin-HRP detection conjugate. After another wash step, the plate was incubated with a chromogenic substrate solution; the reaction was monitored and then stopped by rinsing the plate with water. The number of IFN-γ positive spots in each well of the assay plate was measured using an Immunospot ELISpot reader system.

Immune Phenotype: Cell suspensions were prepared from spleens by mechanical disruption followed by lysis of red blood cells. Tumor cell suspensions were prepared by enzymatic digestion of finely minced tumor slurries. Slurries were incubated in a solution of type IV collagenase (400 units/ml) and DNase 1 (100 μg/ml) for one hour at 37° C. with frequent agitation. Following tumor digestion, debris was separated by sedimentation, and suspensions were passed through a 40 μm nylon cell strainer. Antibody staining of spleen and tumor cell suspensions for FACS analysis was performed following the manufacturer's recommendations (e.g. eBioscience or BD Biosciences).

For the analysis of spleen samples, a parental gate was created around the lymphocyte population as identified by forward and side scatter characteristics. From the lymphocyte gate, subpopulations of immune cells were identified on dot plots: helper T cells (CD4⁺), cytotoxic T lymphocytes (CD8⁺), NK cells (NK1.1⁺), effector memory CD8⁺ T cells (CD8⁺/CD44high/CD62Llow), central memory CD8⁺ T cells (CD8⁺/CD44high/CD62Lhigh) and regulatory T cells (CD4⁺/CD25⁺/Foxp3⁺). To assess degranulation as a measure of lytic activity, CD107a on the lymphocyte cell surface was measured. Following the staining of cell surface proteins, samples were fixed and permeabilized to allow for intracellular staining of the T-box transcription factors (Eomes and T-bet) and effector cytokines (IFN-γ and Granzyme B). From the leukocyte gate, subpopulations of myeloid cells were identified on dot plots: Dendritic cell (CD11c⁺/I-Ab⁺), neutrophils (CD111b⁺/Ly6G⁺), macrophages (CD11b⁺/Ly-6Chigh) and MDSCs (Gr-1+/CD11b⁺).

A similar gating strategy was employed for the analysis of tumor samples, with the exception that a parental gate was first created around the CD45⁺ cell population to identify the tumor infiltrating leukocytes from the other tumor cells and stromal components.

Study Design. TI13-109 Combination Therapy of Radiation with Anti-PD-L1/TGFβ Trap in MC38 Model in B-cell Deficient Mice. Group and treatment (N=10).

### Part 1: Efficacy

| | | | | |
|---|---|---|---|---|
| 1. | Isotype Control | 133 | μg i.v. | day 2 |
| 2. | Radiation | 360 | rads/day | day 0-3 |
| 3. | Anti-PD-L1/TGFβ Trap | 55 | μg i.v. | day 2 |
| 4. | Anti-PD-L1/TGF β Trap | 164 | μg i.v. | day 2 |
| 5. | Radiation | 360 | rads/day | day 0-3 |
| | Anti-PD-L1/TGF β Trap | 55 | μg i.v. | day 2 |
| 6. | Radiation | 360 | rads/day | day 0-3 |
| | Anti- PD-L1/TGF β Trap | 164 | μg i.v. | day 2 |

Part 2: ELISpot Assay: On day 14, all mice were sacrificed and a subset of N=5 mice/group were analyzed for functional responses via ELISpot assay. The spleens were harvested and processed for the ELISpot assay as described above. The number of IFN-γ positive spots in each well of the assay plate was measured using an Immunospot ELISpot reader system.

Study Design: TI14-013 Combination Therapy of Radiation with Anti-PD-L1/TGFβ Trap in MC38 Model in B-cell Deficient Mice. Group and treatment (N=10).

### Part 1: Efficacy

| | | | | |
|---|---|---|---|---|
| 1. | Isotype Control | 45 | μg i.v. | day 2 |
| 2. | Radiation | 360 | rads/day | day 0-3 |
| 3. | Anti-PD-L1/TGFβ Trap | 55 | μg i.v. | day 2 |
| 4. | Radiation | 360 | rads/day | day 0-3 |
| | Anti- PD-L1/TGFβ Trap | 55 | μg i.v. | day 2 |

Part 2: ELISpot Assay and Immune Phenotype. On day 14, all mice were sacrificed and a subset of N=5 mice/group were analyzed for splenic functional responses via ELISpot assay and immune phenotype of TILs. The spleens were harvested and processed for the ELISpot assay as described above. The number of IFN-γ positive spots in each well of the assay plate was measured using an Immunospot ELISpot reader system. Tumor tissue was also harvested and processed as described above. TIL phenotypes were analyzed by FACS analysis for % CD8⁺ TILS, % NK1.1⁺ TILs, CD8⁺ TILs EOMES expression, and CD8⁺ TILs degranulation.

Clinical signs (such as illness and health behavioral changes) were recorded for all animals once daily during the study using the body condition (BC) score system as previously described (Ullman-Cullere and Foltz, Lab Anim Sci. 1999; 49:319-23). Moribund mice were humanely euthanized by CO₂ asphyxiation. Body weights were recorded for all animals on study twice per week, including the termination day of each study. Tumors were measured with digital calipers in three dimensions for the duration of the experiment. Tumor volumes were calculated using the equation: Volume=0.5236 (L×W×H); where L=length, W=width and H=height of the tumor. Kaplan-Meier survival curves were generated to quantify the interval of time from tumor inoculation to sacrifice and calculate the median survival time for each treatment group.

ELISpot assay was used to quantify the frequency of IFN-γ producing, P15E-specific CD8⁺ T cells was quantified by ELISpot assay. Immune phenotype of splenocytes and the tumor infiltrating lymphocytes (TILs) was performed by FACS (Fluorescence-activated cell sorting).

Statistical Analysis: Tumor volumes were measured twice per week throughout the study period. Tumor volume data was presented as the mean±standard error of the mean (SEM). Tumor volume data was log transformed and two-way, repeated measures ANOVA with Tukey's correction for multiple comparisons was performed to measure statistical differences between treatment groups. Tumor weights were collected at study completion. The data was represented as the mean±SEM. The T/C ratio was calculated as the tumor volume (or tumor weight) of the treatment group divided by the tumor volume (or tumor weight) of control group. Tumor weight data was evaluated with one-way ANOVA with Tukey's correction for multiple comparisons to measure statistical differences between treatment groups. The frequency of IFN-γ producing CD8⁺ T cells was quantified by ELISpot assay and represented as the mean number of spots per well (mean±SEM). A one-way ANOVA with Tukey's correction for multiple comparisons was used for statistical analyses using GraphPad Prism Software. p<0.05 was determined to be statistically significant.

Study Design: Combination Therapy of Radiation with Anti-PD-L1/TGFβ Trap in MC38 Model in B-cell Deficient Mice to test abscopal effect. Group and treatment (N=6). Treatment started on day 0 with isotype control (400 days 0, 2, 4), radiation (500 rads/day, days 0, 1, 2, 3), Anti-PD-L1/TGFβ Trap (164 day 0), and Anti-PD-L1/TGFβ Trap (164 day 0)+radiation (500 rads/day, days 0, 1, 2, 3). Radiation was applied only to the primary tumor, as shown in (FIG. 9A). Primary tumor volumes and secondary tumor volumes were measured twice weekly. Tumor volumes are presented as mean±SEM.

### Results

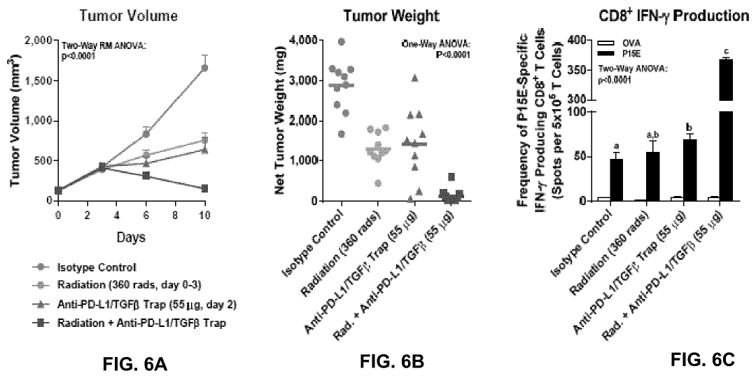

Combination of Radiation with Anti-PD-L1/TGFβ Trap Demonstrated Synergistic Anti-tumor Efficacy. In an MC38 intramuscular tumor model (TI13-109), radiation (360 rads/day, day 0-3) or anti-PD-L1/TGFβ Trap monotherapy (55 or 164 pg, day 2) induced significant tumor growth inhibition (p<0.0001 respectively, vs. isotype control), whereas the combination of radiation and anti-PD-L1/TGFβ Trap induced remarkable therapeutic synergy compared to monotherapy with either radiation (p<0.0001) or anti-PD-L1/TGFβ Trap (p<0.0001) on day 10 (see FIG. 6A). The T/C ratio on day 14 based on the tumor weight was 0.45 for radiation therapy, 0.50 and 0.36 for anti-PD-L1/TGFβ Trap at 55 μg and 164 respectively, and 0.04 vs. 0.01 for the radiation and anti-PD-L1/TGFβ Trap combination groups (55 μg vs. 164 μg, respectively) (see FIG. 6B). Tumor regression was observed, as early as 4 days after the anti-PD-L1/TGFβ Trap treatment, in 50% (10 out of 20) of the mice treated with anti-PD-L1/TGFβ Trap monotherapy, 100% (20 out of 20) of the mice treated with the combination therapy, and only 10% (1 out of 10) of the mice treated with radiation monotherapy. Furthermore, since anti-PD-L1/TGFβ Trap was given only as a single dose, 4 of 10 regressed tumors grew back in the anti-PD-L1/TGFβ Trap monotherapy group, whereas all the regressed tumors in the combination group continued to shrink up to day 14 when the mice were sacrificed for evaluation of immune function.

Immune Activation Was Correlated With the Antitumor Efficacy. On day 14, the mice were sacrificed and the frequency of IFN-γ producing, tumor-reactive (P15E) CD8⁺ T cells was quantified using an ex vivo ELISpot assay (see FIG. 6C). Only a moderate induction of IFN-γ producing tumor-reactive CD8⁺ T cells was observed in the radiation and anti-PD-L1/TGFβ Trap monotherapy group (p>0.05 and p<0.05 respectively, vs. isotype control). Consistent with the observed antitumor efficacy; however, mice treated with the combination therapy experienced a synergistic induction in the frequency P15E-specific, IFN-γ producing CD8⁺ T cells (see FIG. 6C). The CD8⁺ T cell IFN-γ production induced by combination therapy was 7-fold above that of the isotype control and at least 5-fold above those of the monotherapies (p<0.001 vs. each monotherapy, respectively). In this study (TI13-109), increasing the dose of anti-PD-L1/TGFβ Trap from 55 pg to 164 pg in the combination therapy did not further accelerate tumor regression. Due to a low CD8⁺ T cell yield in the high dose group, an adequate evaluation of the frequency of IFN-γ producing, tumor reactive (P15E) CD8⁺ T cells could not be performed. Therefore, a repeat study was performed to ensure the consistency of the findings.

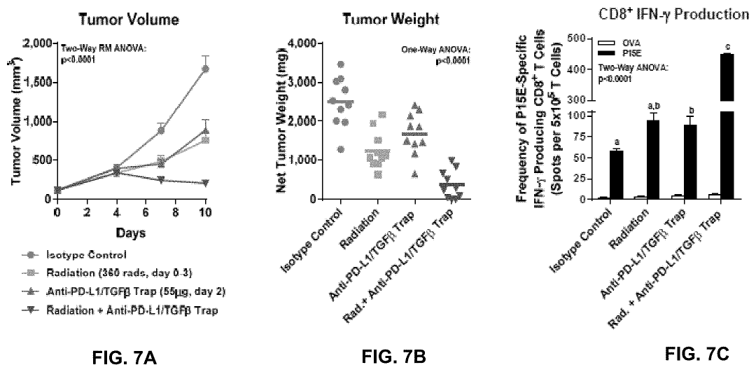

A repeat experiment (TI14-013) with the low dose of 55 μg of anti-PD-L1/TGFβ Trap yielded nearly identical synergistic effects on both tumor growth inhibition and induction of P15E specific CD8⁺ T cell IFN-γ production with the combination therapy (see FIG. 7A-C).

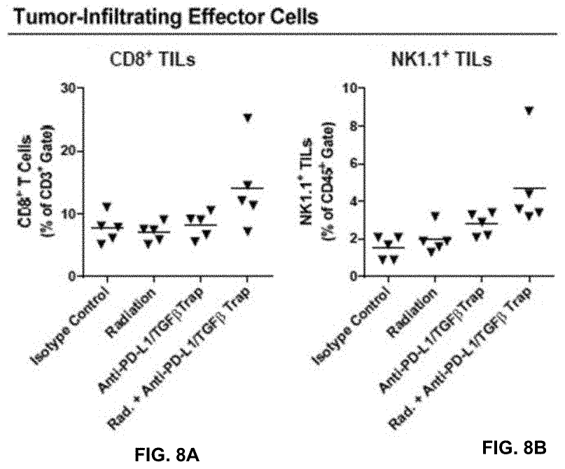

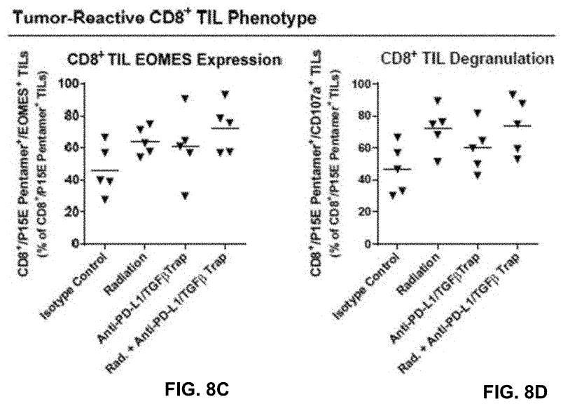

An analysis of the tumor-infiltrating lymphocytes of mice treated in study TI14-013 revealed elevations in the frequencies of CD8⁺ TILs and NK cells following treatment with the combination of radiation and anti-PD-L1/TGFβ Trap relative to either monotherapy or the Isotype control groups (see FIG. 8A-B). Additional analysis indicated that the combination of radiation and anti-PD-L1/TGFβ Trap therapy promoted the expression of the T-box transcription factor, Eomes, and degranulation (CD107a) on tumor-infiltrating CD8⁺ T cells (see FIG. 8C-D).

Combination therapy with radiation and a single dose of anti-PD-L1/TGFβ Trap reduced primary tumor volume relative to anti-PD-L1/TGFβ Trap or radiation alone (p<0.0001 for both, day 14) (FIG. 9B). However, combination therapy also reduced secondary tumor volume relative to anti-PD-L1/TGFβ Trap or radiation alone (p=0.0066 and p=0.0006, respectively, day 14) (FIG. 9C). Notably, neither radiation alone nor a single low dose of anti-PD-L1/TGFβ Trap significantly inhibited secondary tumor growth relative to isotype control treatment, indicating anti-PD-L1/TGFβ Trap synergized with radiation to induce an abscopal effect.

The observed synergies in antitumor efficacy and the frequencies of tumor-reactive, IFN-γ producing CD8⁺ T cells coupled with enhanced infiltration by effector CD8⁺ T cells and NK cells is consistent with the profound induction of innate and adaptive antitumor immune responses by combination therapy with radiation and the anti-PD-L1/TGFβ Trap molecule. As such, this therapeutic combination has clinically relevant applications for improving radiotherapy in cancer patients.

The data described herein demonstrate that standard of care external beam radiation therapy (EBRT) can be combined with anti-PD-L1/TGFβ Trap therapy to achieve synergistic tumor growth inhibition and the induction of tumor-reactive CD8⁺ T cell responses in a MC38 colorectal cancer model. As P15E is an endogenous retroviral antigen expressed by the MC38 tumor cell line (Zeh et al. J Immunol. 1999; 162:989-94), the observed increase in P15E-specific, IFN-γ producing CD8⁺ T cells constitutes a tumor-reactive, and not a generalized, T cell response following combination therapy. This synergistic immunological response is consistent with the enhanced antitumor efficacy observed in this therapeutic regimen, indicating that the combined treatment with radiation and anti-PD-L1/TGFβ Trap facilitates a CD8⁺ T cell mediated antitumor response. The combination of radiation and anti-PD-L1/TGFβ Trap therapy were also shown to promote MC38 tumor infiltration by CD8⁺ T cells and NK cells. Furthermore, the combination therapy induced an effector CD8⁺ TIL phenotype as evidenced by higher expression levels of the transcription factor, Eomes, and the degranulation marker, CD107a.

The observed results support the synergy of this combination strategy for potential clinical application. The sequential therapy of radiation and anti-PD-L1/TGFβ Trap can be of benefit to patients who have increased circulating TGFβ levels following radiotherapy. Furthermore, because of the strong synergistic effect observed even with a single low dose of anti-PD-L1/TGFβ Trap, a favorable safety profile in the clinic with such a combination therapy is expected.

SEQUENCES
Peptide sequence of the secreted anti-PD-L1 lambda light chain
SEQ ID NO: 1
QSALTQPASVSGSPGQSITISCTGTSSDVGGYNYVSWYQQHPGKAPKLMIYDVSNRPSGVSNRF
SGSKSGNTASLTISGLQAEDEADYYCSSYTSSSTRVFGTGTKVTVLGQPKANPTVTLFPPSSEE
LQANKATLVCLISDFYPGAVTVAWKADGSPVKAGVETTKPSKQSNNKYAASSYLSLTPEQWKSH
RSYSCQVTHEGSTVEKTVAPTECS
Peptide sequence of the secreted H chain of anti-PDL1
SEQ ID NO: 2
EVQLLESGGGLVQPGGSLRLSCAASGFTFSSYIMMWVRQAPGKGLEWVSSIYPSGGITFYADTV
KGRFTISRDNSKNTLYLQMNSLRAEDTAVYYCARIKLGTVTTVDYWGQGTLVTVSSASTKGPSV
FPLAPSSKSTSGGTAALGCLVKDYFPEPVTVSWNSGALTSGVHTFPAVLQSSGLYSLSSVVTVP
SSSLGTQTYICNVNHKPSNTKVDKRVEPKSCDKTHTCPPCPAPELLGGPSVFLFPPKPKDTLMI
SRTPEVTCVVVDVSHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVMHEALHNHYTQKSLSLSP
GK
Peptide sequence of the secreted H chain of anti-PDL1/TGFβ Trap
SEQ ID NO: 3
EVQLLESGGGLVQPGGSLRLSCAASGFTFSSYIMMWVRQAPGKGLEWVSSIYPSGGITFYADTV
KGRFTISRDNSKNTLYLQMNSLRAEDTAVYYCARIKLGTVTTVDYWGQGTLVTVSSASTKGPSV
FPLAPSSKSTSGGTAALGCLVKDYFPEPVTVSWNSGALTSGVHTFPAVLQSSGLYSLSSVVTVP
SSSLGTQTYICNVNHKPSNTKVDKRVEPKSCDKTHTCPPCPAPELLGGPSVFLFPPKPKDTLMI
SRTPEVTCVVVDVSHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVMHEALHNHYTQKSLSLSP
GAGGGGSGGGGSGGGGSGGGGSGIPPHVQKSVNNDMIVTDNNGAVKFPQLCKFCDVRFSTCDNQ
KSCMSNCSITSICEKPQEVCVAVWRKNDENITLETVCHDPKLPYHDFILEDAASPKCIMKEKKK
PGETFFMCSCSSDECNDNIIFSEEYNTSNPD
DNA sequence from the translation initiation codon to the
translation stop codon of the anti-PD-L1 lambda light chain (the
leader sequence preceding the VL is the signal peptide from
urokinase plasminogen activator)
SEQ ID NO: 4
atgagggccctgctggctagactgctgctgtgcgtgctggtcgtgtccgacagcaagggcCAGT
CCGCCCTGACCCAGCCTGCCTCCGTGTCTGGCTCCCCTGGCCAGTCCATCACCATCAGCTGCAC
CGGCACCTCCAGCGACGTGGGCGGCTACAACTACGTGTCCTGGTATCAGCAGCACCCCGGCAAG
GCCCCCAAGCTGATGATCTACGACGTGTCCAACCGGCCCTCCGGCGTGTCCAACAGATTCTCCG
GCTCCAAGTCCGGCAACACCGCCTCCCTGACCATCAGCGGACTGCAGGCAGAGGACGAGGCCGA
CTACTACTGCTCCTCCTACACCTCCTCCAGCACCAGAGTGTTCGGCACCGGCACAAAAGTGACC
GTGCTGggccagcccaaggccaacccaaccgtgacactgttccccccatcctccgaggaactgc
aggccaacaaggccaccctggtctgcctgatctcagatttctatccaggcgccgtgaccgtggc
ctggaaggctgatggctccccagtgaaggccggcgtggaaaccaccaagccctccaagcagtcc
aacaacaaatacgccgcctcctcctacctgtccctgacccccgagcagtggaagtcccaccggt
cctacagctgccaggtcacacacgagggctccaccgtggaaaagaccgtcgcccccaccgagtg
ctcaTGA
DNA sequence from the translation initiation codon to the
translation stop codon (mVK SP leader: small underlined; VH:
capitals; IgG1m3 with K to A mutation: small letters;
(G4S)x4-G linker (SEQ ID NO: 11): bold capital letters;
TGFβRII: bold underlined small letters; two stop codons: bold
underlined capital letters)
SEQ ID NO: 5
atggaaacagacaccctgctgctgtgggtgctgctgctgtgggtgcccggctccacaggcGAGG
TGCAGCTGCTGGAATCCGGCGGAGGACTGGTGCAGCCTGGCGGCTCCCTGAGACTGTCTTGCGC
CGCCTCCGGCTTCACCTTCTCCAGCTACATCATGATGTGGGTGCGACAGGCCCCTGGCAAGGGC
CTGGAATGGGTGTCCTCCATCTACCCCTCCGGCGGCATCACCTTCTACGCCGACACCGTGAAGG
GCCGGTTCACCATCTCCCGGGACAACTCCAAGAACACCCTGTACCTGCAGATGAACTCCCTGCG
GGCCGAGGACACCGCCGTGTACTACTGCGCCCGGATCAAGCTGGGCACCGTGACCACCGTGGAC
TACTGGGGCCAGGGCACCCTGGTGACAGTGTCCTCCgctagcaccaagggcccatcggtcttcc
ccctggcaccctcctccaagagcacctctgggggcacagcggccctgggctgcctggtcaagga
ctacttccccgaaccggtgacggtgtcgtggaactcaggcgccctgaccagcggcgtgcacacc
ttcccggctgtcctacagtcctcaggactctactccctcagcagcgtggtgaccgtgccctcca
gcagcttgggcacccagacctacatctgcaacgtgaatcacaagcccagcaacaccaaggtgga
caagagagttgagcccaaatcttgtgacaaaactcacacatgcccaccgtgcccagcacctgaa
ctcctggggggaccgtcagtcttcctcttccccccaaaacccaaggacaccctcatgatctccc
ggacccctgaggtcacatgcgtggtggtggacgtgagccacgaagaccctgaggtcaagttcaa
ctggtacgtggacggcgtggaggtgcataatgccaagacaaagccgcgggaggagcagtacaac
agcacgtaccgtgtggtcagcgtcctcaccgtcctgcaccaggactggctgaatggcaaggagt
acaagtgcaaggtctccaacaaagccctcccagcccccatcgagaaaaccatctccaaagccaa
agggcagccccgagaaccacaggtgtacaccctgcccccatcccgggaggagatgaccaagaac
caggtcagcctgacctgcctggtcaaaggcttctatcccagcgacatcgccgtggagtgggaga
gcaatgggcagccggagaacaactacaagaccacgcctcccgtgctggactccgacggctcctt
cttcctctatagcaagctcaccgtggacaagagcaggtggcagcaggggaacgtcttctcatgc
tccgtgatgcatgaggctctgcacaaccactacacgcagaagagcctctccctgtccccgggtg
ctGGCGGCGGAGGAAGCGGAGGAGGTGGCAGCGGTGGCGGTGGCTCCGGCGGAGGTGGCTCCGG
Aatccctccccacgtgcagaagtccgtgaacaacgacatgatcgtgaccgacaacaacggcgcc
gtgaagttccctcagctgtgcaagttctgcgacgtgaggttcagcacctgcgacaaccagaagt
cctgcatgagcaactgcagcatcacaagcatctgcgagaagccccaggaggtgtgtgtggccgt
gtggaggaagaacgacgaaaacatcaccctcgagaccgtgtgccatgaccccaagctgccctac
cacgacttcatcctggaagacgccgcctcccccaagtgcatcatgaaggagaagaagaagcccg
gcgagaccttcttcatgtgcagctgcagcagcgacgagtgcaatgacaacatcatctttagcga
ggagtacaacaccagcaaccccgacTGATAA
Polypeptide sequence of the secreted lambda light chain of
anti-PD-L1 (mut)/TGFβ Trap, with mutations A31G, D52E, R99Y
SEQ ID NO: 6
QSALTQPASVSGSPGQSITISCTGTSSDVGGYNYVSWYQQHPGKAPKLMIYEVSNRPSGVSNRF
SGSKSGNTASLTISGLQAEDEADYYCSSYTSSSTYVFGTGTKVTVLGQPKANPTVTLFPPSSEE
LQANKATLVCLISDFYPGAVTVAWKADGSPVKAGVETTKPSKQSNNKYAASSYLSLTPEQWKSH
RSYSCQVTHEGSTVEKTVAPTECS
Polypeptide sequence of the secreted heavy chain of anti-PD-L1
(mut)/TGFβ Trap
SEQ ID NO: 7
EVQLLESGGGLVQPGGSLRLSCAASGFTFSMYMKMWVRQAPGKGLEWVSSIYPSGGITFYADSV
KGRFTISRDNSKNTLYLQMNSLRAEDTAIYYCARIKLGTVTTVDYWGQGTLVTVSSASTKGPSV
FPLAPSSKSTSGGTAALGCLVKDYFPEPVTVSWNSGALTSGVHTFPAVLQSSGLYSLSSVVTVP
SSSLGTQTYICNVNHKPSNTKVDKRVEPKSCDKTHTCPPCPAPELLGGPSVFLFPPKPKDTLMI
SRTPEVTCVVVDVSHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVMHEALHNHYTQKSLSLSP
GAGGGGSGGGGSGGGGSGGGGSGIPPHVQKSVNNDMIVTDNNGAVKFPQLCKFCDVRFSTCDNQ
KSCMSNCSITSICEKPQEVCVAVWRKNDENITLETVCHDPKLPYHDFILEDAASPKCIMKEKKK
PGETFFMCSCSSDECNDNIIFSEEYNTSNPD
Human TGFβRII Isoform A Precursor Polypeptide (NCBI RefSeq
Accession No: NP_001020018)
SEQ ID NO: 8
MGRGLLRGLWPLHIVLWTRIASTIPPHVQKSDVEMEAQKDEIICPSCNRTAHPLRHINNDMIVT
DNNGAVKFPQLCKFCDVRFSTCDNQKSCMSNCSITSICEKPQEVCVAVWRKNDENITLETVCHD
PKLPYHDFILEDAASPKCIMKEKKKPGETFFMCSCSSDECNDNIIFSEEYNTSNPDLLLVIFQV
TGISLLPPLGVAISVIIIFYCYRVNRQQKLSSTWETGKTRKLMEFSEHCAIILEDDRSDISSTC
ANNINHNTELLPIELDTLVGKGRFAEVYKAKLKQNTSEQFETVAVKIFPYEEYASWKTEKDIFS
DINLKHENILQFLTAEERKTELGKQYWLITAFHAKGNLQEYLTRHVISWEDLRKLGSSLARGIA
HLHSDHTPCGRPKMPIVHRDLKSSNILVKNDLTCCLCDFGLSLRLDPTLSVDDLANSGQVGTAR
YMAPEVLESRMNLENVESFKQTDVYSMALVLWEMTSRCNAVGEVKDYEPPFGSKVREHPCVESM
KDNVLRDRGRPEIPSFWLNHQGIQMVCETLTECWDHDPEARLTAQCVAERFSELEHLDRLSGRS
CSEEKIPEDGSLNTTK
Human TGFβRII Isoform B Precursor Polypeptide (NCBI RefSeq
Accession No: NP_003233
SEQ ID NO: 9
MGRGLLRGLWPLHIVLWTRIASTIPPHVQKSVNNDMIVTDNNGAVKFPQLCKFCDVRFSTCDNQ
KSCMSNCSITSICEKPQEVCVAVWRKNDENITLETVCHDPKLPYHDFILEDAASPKCIMKEKKK
PGETFFMCSCSSDECNDNIIFSEEYNTSNPDLLLVIFQVTGISLLPPLGVAISVIIIFYCYRVN
RQQKLSSTWETGKTRKLMEFSEHCAIILEDDRSDISSTCANNINHNTELLPIELDTLVGKGRFA
EVYKAKLKQNTSEQFETVAVKIFPYEEYASWKTEKDIFSDINLKHENILQFLTAEERKTELGKQ
YWLITAFHAKGNLQEYLTRHVISWEDLRKLGSSLARGIAHLHSDHTPCGRPKMPIVHRDLKSSN
ILVKNDLTCCLCDFGLSLRLDPTLSVDDLANSGQVGTARYMAPEVLESRMNLENVESFKQTDVY
SMALVLWEMTSRCNAVGEVKDYEPPFGSKVREHPCVESMKDNVLRDRGRPEIPSFWLNHQGIQM
VCETLTECWDHDPEARLTAQCVAERFSELEHLDRLSGRSCSEEKIPEDGSLNTTK
A Human TGFβRII Isoform B Extracellular Domain Polypeptide
SEQ ID NO: 10
IPPHVQKSVNNDMIVTDNNGAVKFPQLCKFCDVRFSTCDNQKSCMSNCSITSICEKPQEVCVAV
WRKNDENITLETVCHDPKLPYHDFILEDAASPKCIMKEKKKPGETFFMCSCSSDECNDNIIFSE
EYNTSNPD
(Gly₄Ser)₄Gly linker
SEQ ID NO: 11
GGGGSGGGGSGGGGSGGGGSG
Polypeptide sequence of the secreted heavy chain variable
region of anti-PD-L1 antibody MPDL3280A
SEQ ID NO: 12
EVQLVESGGGLVQPGGSLRLSCAASGFTFSDSWIHWVRQAPGKGLEWVAWISPYGGSTYY
ADSVKGRFTISADTSKNTAYLQMNSLRAEDTAVYYCARRHWPGGFDYWGQGTLVTVSS
Polypeptide sequence of the secreted light chain variable region
of anti-PD-L1 antibody MPDL3280A and the anti-PD-L1 antibody
YW243.55S70
SEQ ID NO: 13
DIQMTQSPSSLSASVGDRVTITCRASQDVSTAVAWYQQKPGKAPKLLIYSASFLYSGVPSRFSG
SGSGTDFTLTISSLQPEDFATYYCQQYLYHPATFGQGTKVEIKR
Polypeptide sequence of the secreted heavy chain variable region
of anti-PD-L1 antibody YW243.55S70
SEQ ID NO: 14
EVQLVESGGGLVQPGGSLRLSCAASGFTFSDSWIHWVRQAPGKGLEWVAWISPYGGSTYYADSV
KGRFTISADTSKNTAYLQMNSLRAEDTAVYYCARRHWPGGFDYWGQGTLVTVSA

### INCORPORATION BY REFERENCE

The entire disclosure of each of the patent documents and scientific articles referred to herein is incorporated by reference for all purposes. The entire disclosure of U.S. application Ser. No. 14/618,454 is incorporated by reference herein in its entirety for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Various structural elements of the different embodiments and various disclosed method steps may be utilized in various combinations and permutations, and all such variants are to be considered forms of the invention. Scope of the invention is thus indicated by the appended claims rather than by the foregoing description, and all changes that come within the meaning and range of equivalency of the claims are intended to be embraced therein.

1. A method of treating cancer, the method comprising administering to a cancer patient:
(i) a protein comprising a human TGFβRII, or fragment thereof capable of binding TGFβ; and an antibody, or antigen-binding fragment thereof, that binds human protein Programmed Death Ligand 1 (PD-L1); and
(ii) an effective amount of at least one additional anti-cancer agent,
thereby providing a combination therapy having an enhanced therapeutic effect compared to the effect of the protein and the at least one additional anti-cancer agent each administered alone.

2. A method of inhibiting tumor growth, the method comprising exposing the tumor to:
(i) a protein comprising a first moiety comprising a human TGFβRII, or fragment thereof capable of binding TGFβ, and an antibody, or antigen-binding fragment thereof, that binds human protein Programmed Death Ligand 1 (PD-L1); and
(ii) an effective amount of at least one additional anti-cancer agent,
thereby providing a combination therapy having an enhanced therapeutic effect compared to the effect of the protein and the at least one additional anti-cancer agent each administered alone.

3. The method of claim 1, wherein the antibody, or antigen-binding fragment thereof, that binds PD-L1 comprises amino acids 1-120 of SEQ ID NO:2.

4. The method of claim 1, wherein the antibody, or antigen-binding fragment thereof, that binds PD-L1 comprises the amino acid sequence of SEQ ID NO:2 except that the C-terminal lysine has been mutated to alanine.

5. The method of claim 1, wherein the antibody, or antigen-binding fragment thereof, that binds PD-L1 comprises the amino acid sequences SYIMM (SEQ ID NO: 34) (HVR-H1), SIYPSGGITFYADTVKG (SEQ ID NO: 35) (HVR-H2) and IKLGTVTTVDY (SEQ ID NO: 36) (HVR-H3).

6. The method of claim 1, wherein the human TGFβRII, or fragment thereof capable of binding TGFβ comprises the amino acid sequence of SEQ ID NO:10.

7. The method of claim 1, wherein the protein comprises the amino acid sequence of SEQ ID NO:1 and SEQ ID NO:3.

8. The method of claim 1, where the anti-cancer agent is a chemotherapeutic agent.

9. The method of claim 1, wherein the anti-cancer agent is radiation.

10. The method of claim 8, wherein the chemotherapeutic agent is an alkylating agent.

11. (canceled)

12. The method of claim 8, wherein the chemotherapeutic agent is a platinum-based agent.

13. (canceled)

14. The method of claim 1, wherein the cancer is selected from the group consisting of colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, non-small cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodysplastic syndromes.

15. The method of claim 8, wherein the administration of an initial dose of chemotherapy is followed by administration of the protein.

16. The method of claim 9, wherein the administration of an initial dose of radiation is followed by administration of the protein.

17. (canceled)

18. The method of claim 9, wherein multiple doses of radiation are administered.

19. (canceled)

20. The method of claim 1, wherein the dosage of the protein is selected from the group consisting of (i) a dosage known to be used for treatment of said cancer and (ii) a lower dosage compared to the concentration known to be used for treating said cancer.

21. The method of claim 8, wherein the dosage of the chemotherapeutic agent is selected from the group consisting of (i) a dosage known to be used for treatment of said cancer and (ii) a lower dosage compared to the concentration known to be used for treating said cancer.

22. The method of claim 9, wherein the dosage of the radiation is selected from the group consisting of (i) a dosage known to be used for treatment of said cancer and (ii) a lower dosage compared to the concentration known to be used for treating said cancer.

23. (canceled)

24. The method of claim 1, wherein the protein and one additional anti-cancer agent are administered sequentially.

25. The method of claim 9, wherein the method inhibits the growth of a secondary tumor or metastasis distal to the primary tumor treated with radiation.


SEQUENCE LISTING (50 sequences)

SEQ ID NO: 1 - protein, 216 aa, Artificial Sequence
  source: /note=“Description of Artificial Sequence Synthetic polypeptide”
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 2 - protein, 450 aa, Artificial Sequence
  source: /note=“Description of Artificial Sequence Synthetic polypeptide”
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 607 aa, Artificial Sequence
  source: /note=“Description of Artificial Sequence Synthetic polypeptide”
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 4 - DNA, 711 bp, Artificial Sequence
  source: /note=“Description of Artificial Sequence Synthetic polynucleotide”
atgagggccctgctggctagactgctgctgtgcgtgctggtcgtgtccgacagcaagggccagtccgccctgacccagcctgcctccgtgtctggctcccctggccagtccatcaccatcagctgcaccggcacctccagcgacgtgggcggctacaactacgtgtcctggtatcagcagcaccccggcaaggcccccaagctgatgatctacgacgtgtccaaccggccctccggcgtgtccaacagattctccggctccaagtccggcaacaccgcctccctgaccatcagcggactgcaggcagaggacgaggccgactactactgctcctcctacacctcctccagcaccagagtgttcggcaccggcacaaaagtgaccgtgctgggccagcccaaggccaacccaaccgtgacactgttccccccatcctccgaggaactgcaggccaacaaggccaccctggtctgcctgatctcagatttctatccaggcgccgtgaccgtggcctggaaggctgatggctccccagtgaaggccggcgtggaaaccaccaagccctccaagcagtccaacaacaaatacgccgcctcctcctacctgtccctgacccccgagcagtggaagtcccaccggtcctacagctgccaggtcacacacgagggctccaccgtggaaaagaccgtcgcccccaccgagtgctcatga

SEQ ID NO: 5 - DNA, 1887 bp, Artificial Sequence
  source: /note=“Description of Artificial Sequence Synthetic polynucleotide”
atggaaacagacaccctgctgctgtgggtgctgctgctgtgggtgcccggctccacaggcgaggtgcagctgctggaatccggcggaggactggtgcagcctggcggctccctgagactgtcttgcgccgcctccggcttcaccttctccagctacatcatgatgtgggtgcgacaggcccctggcaagggcctggaatgggtgtcctccatctacccctccggcggcatcaccttctacgccgacaccgtgaagggccggttcaccatctcccgggacaactccaagaacaccctgtacctgcagatgaactccctgcgggccgaggacaccgccgtgtactactgcgcccggatcaagctgggcaccgtgaccaccgtggactactggggccagggcaccctggtgacagtgtcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtgctggcggcggaggaagcggaggaggtggcagcggtggcggtggctccggcggaggtggctccggaatccctccccacgtgcagaagtccgtgaacaacgacatgatcgtgaccgacaacaacggcgccgtgaagttccctcagctgtgcaagttctgcgacgtgaggttcagcacctgcgacaaccagaagtcctgcatgagcaactgcagcatcacaagcatctgcgagaagccccaggaggtgtgtgtggccgtgtggaggaagaacgacgaaaacatcaccctcgagaccgtgtgccatgaccccaagctgccctaccacgacttcatcctggaagacgccgcctcccccaagtgcatcatgaaggagaagaagaagcccggcgagaccttcttcatgtgcagctgcagcagcgacgagtgcaatgacaacatcatctttagcgaggagtacaacaccagcaaccccgactgataa

SEQ ID NO: 6 - protein, 216 aa, Artificial Sequence
  source: /note=“Description of Artificial Sequence Synthetic polypeptide”
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTYVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 7 - protein, 607 aa, Artificial Sequence
  source: /note=“Description of Artificial Sequence Synthetic polypeptide”
EVQLLESGGGLVQPGGSLRLSCAASGFTFSMYMMMWVRQAPGKGLEWVSSIYPSGGITFYADSVKGRFTISRDNSKNTLYLQMNSLRAEDTAIYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 8 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 9 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 10 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 11 - protein, 21 aa, Artificial Sequence
  source: /note=“Description of Artificial Sequence Synthetic peptide”
GGGGSGGGGSGGGGSGGGGSG

SEQ ID NO: 12 - protein, 118 aa, Artificial Sequence [sequence omitted]

SEQ ID NO: 13 - protein, 108 aa, Artificial Sequence
  source: /note=“Description of Artificial Sequence Synthetic polypeptide”
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 14 - protein, 118 aa, Artificial Sequence
  source: /note=“Description of Artificial Sequence Synthetic polypeptide”
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 15 - protein, 4 aa, Artificial Sequence
  source: /note=“Description of Artificial Sequence Synthetic peptide”
QFNS

SEQ ID NO: 16 - protein, 4 aa, Artificial Sequence
  source: /note=“Description of Artificial Sequence Synthetic peptide”
QAQS

SEQ ID NO: 17 - protein, 6 aa, Artificial Sequence
  source: /note=“Description of Artificial Sequence Synthetic peptide”
PKSCDK

SEQ ID NO: 18 - protein, 6 aa, Artificial Sequence
  source: /note=“Description of Artificial Sequence Synthetic peptide”
PKSSDK

SEQ ID NO: 19 - protein, 4 aa, Artificial Sequence
  source: /note=“Description of Artificial Sequence Synthetic peptide”
LSLS

SEQ ID NO: 20 - protein, 4 aa, Artificial Sequence
  source: /note=“Description of Artificial Sequence Synthetic peptide”
ATAT

SEQ ID NO: 21 - protein, 17 aa, Artificial Sequence
  source: /note=“Description of Artificial Sequence Synthetic peptide”
  MOD_RES (8)..(8): /replace=“Ile”
  MOD_RES (14)..(14): /replace=“Thr”
  MISC_FEATURE (1)..(17): /note=“Variant residues given in the sequence have no preference with respect to those in the annotations for variant positions”
SIYPSGGFTFYADSVKG

SEQ ID NO: 22 - protein, 11 aa, Artificial Sequence
  source: /note=“Description of Artificial Sequence Synthetic peptide”
  MOD_RES (10)..(10): /replace=“Asp”
  MISC_FEATURE (1)..(11): /note=“Variant residues given in the sequence have no preference with respect to those in the annotations for variant positions”
IKLGTVTTVEY

SEQ ID NO: 23 - protein, 30 aa, Artificial Sequence
  source: /note=“Description of Artificial Sequence Synthetic polypeptide”
EVQLLESGGGLVQPGGSLRLSCAASGFTFS

SEQ ID NO: 24 - protein, 14 aa, Artificial Sequence
  source: /note=“Description of Artificial Sequence Synthetic peptide”
WVRQAPGKGLEWVS

SEQ ID NO: 25 - protein, 32 aa, Artificial Sequence
  source: /note=“Description of Artificial Sequence Synthetic polypeptide”
RFTISRDNSKNTLYLQMNSLRAEDTAVYYCAR

SEQ ID NO: 26 - protein, 11 aa, Artificial Sequence
  source: /note=“Description of Artificial Sequence Synthetic peptide”
WGQGTLVTVSS

SEQ ID NO: 27 - protein, 14 aa, Artificial Sequence
  source: /note=“Description of Artificial Sequence Synthetic peptide”
  MOD_RES (4)..(4): /replace=“Ser”
  MOD_RES (5)..(5): /replace=“Arg” or “Ser”
  MOD_RES (9)..(9): /replace=“Gly”
  MISC_FEATURE (1)..(14): /note=“Variant residues given in the sequence have no preference with respect to those in the annotations for variant positions”
TGTNTDVGAYNYVS

SEQ ID NO: 28 - protein, 7 aa, Artificial Sequence
  source: /note=“Description of Artificial Sequence Synthetic peptide”
  MOD_RES (1)..(1): /replace=“Asp”
  MOD_RES (3)..(3): /replace=“Asn” or “Ser”
  MOD_RES (4)..(4): /replace=“His” or “Asn”
  MISC_FEATURE (1)..(7): /note=“Variant residues given in the sequence have no preference with respect to those in the annotations for variant positions”
EVIDRPS

SEQ ID NO: 29 - protein, 10 aa, Artificial Sequence
  source: /note=“Description of Artificial Sequence Synthetic peptide”
  MOD_RES (3)..(3): /replace=“Tyr”
  MOD_RES (5)..(5): /replace=“Ser”
  MOD_RES (6)..(6): /replace=“Thr” or “Ser”
  MOD_RES (7)..(7): /replace=“Ser”
  MOD_RES (8)..(8): /replace=“Thr”
  MISC_FEATURE (1)..(10): /note=“Variant residues given in the sequence have no preference with respect to those in the annotations for variant positions”
SSFTNRGIRV

SEQ ID NO: 30 - protein, 22 aa, Artificial Sequence
  source: /note=“Description of Artificial Sequence Synthetic peptide”
QSALTQPASVSGSPGQSITISC

SEQ ID NO: 31 - protein, 15 aa, Artificial Sequence
  source: /note=“Description of Artificial Sequence Synthetic peptide”
WYQQHPGKAPKLMIY

SEQ ID NO: 32 - protein, 32 aa, Artificial Sequence
  source: /note=“Description of Artificial Sequence Synthetic polypeptide”
GVSNRFSGSKSGNTASLTISGLQAEDEADYYC

SEQ ID NO: 33 - protein, 10 aa, Artificial Sequence
  source: /note=“Description of Artificial Sequence Synthetic peptide”
FGTGTKVTVL

SEQ ID NO: 34 - protein, 5 aa, Artificial Sequence
  source: /note=“Description of Artificial Sequence Synthetic peptide”
SYIMM

SEQ ID NO: 35 - protein, 17 aa, Artificial Sequence
  source: /note=“Description of Artificial Sequence Synthetic peptide”
SIYPSGGITFYADTVKG

SEQ ID NO: 36 - protein, 11 aa, Artificial Sequence
  source: /note=“Description of Artificial Sequence Synthetic peptide”
IKLGTVTTVDY

SEQ ID NO: 37 - protein, 14 aa, Artificial Sequence
  source: /note=“Description of Artificial Sequence Synthetic peptide”
TGTSSDVGGYNYVS

SEQ ID NO: 38 - protein, 7 aa, Artificial Sequence
  source: /note=“Description of Artificial Sequence Synthetic peptide”
DVSNRPS

SEQ ID NO: 39 - protein, 10 aa, Artificial Sequence
  source: /note=“Description of Artificial Sequence Synthetic peptide”
SSYTSSSTRV

SEQ ID NO: 40 - protein, 5 aa, Artificial Sequence
  source: /note=“Description of Artificial Sequence Synthetic peptide”
MYMMM

SEQ ID NO: 41 - protein, 17 aa, Artificial Sequence
  source: /note=“Description of Artificial Sequence Synthetic peptide”
SIYPSGGITFYADSVKG

SEQ ID NO: 42 - protein, 14 aa, Artificial Sequence
  source: /note=“Description of Artificial Sequence Synthetic peptide”
TGTSSDVGAYNYVS

SEQ ID NO: 43 - protein, 119 aa, Artificial Sequence
  source: /note=“Description of Artificial Sequence Synthetic polypeptide”
EVQLLESGGGLVQPGGSLRLSCAASGFTFSSYIMMVWRQAPGKGLEWVSSIYPSGGITFYADWKGRFTISRDNSKNTLYLQMNSLRAEDTAVYYCARIKLGTVTTVDYWGQGTLVTVSS

SEQ ID NO: 44 - protein, 110 aa, Artificial Sequence
  source: /note=“Description of Artificial Sequence Synthetic polypeptide”
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 45 - protein, 120 aa, Artificial Sequence
  source: /note=“Description of Artificial Sequence Synthetic polypeptide”
EVQLLESGGGLVQPGGSLRLSCAASGFTFSMYMMMWVRQAPGKGLEVWSSIYPSGGITFYADSVKGRFTISRDNSKNTLYLQMNSLRAEDTAIYYCARIKLGTVTTVDYWGQGTLVTVSS

SEQ ID NO: 46 - protein, 110 aa, Artificial Sequence
  source: /note=“Description of Artificial Sequence Synthetic polypeptide”
QSALTQPASVSGSPGQSITISCTGTSSDVGAYNYVSWYQQHPGKAPKLMIYDVSNRPSGVSNRFSGSKSGNTASLTISGLQAEDEADYYCSSYTSSSTRVFGTGTKVTVL

SEQ ID NO: 47 - DNA, 1407 bp, Artificial Sequence
  source: /note=“Description of Artificial Sequence Synthetic polynucleotide”
atggagttgcctgttaggctgttggtgctgatgttctggattcctgctagctccagcgaggtgcagctgctggaatccggcggaggactggtgcagcctggcggctccctgagactgtcttgcgccgcctccggcttcaccttctccagctacatcatgatgtgggtgcgacaggcccctggcaagggcctggaatgggtgtcctccatctacccctccggcggcatcaccttctacgccgacaccgtgaagggccggttcaccatctcccgggacaactccaagaacaccctgtacctgcagatgaactccctgcgggccgaggacaccgccgtgtactactgcgcccggatcaagctgggcaccgtgaccaccgtggactactggggccagggcaccctggtgacagtgtcctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcacgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 48 - DNA, 705 bp, Artificial Sequence
  source: /note=“Description of Artificial Sequence Synthetic polynucleotide”
atggagttgcctgttaggctgttggtgctgatgttctggattcctgcttccttaagccagtccgccctgacccagcctgcctccgtgtctggctcccctggccagtccatcaccatcagctgcaccggcacctccagcgacgtgggcggctacaactacgtgtcctggtatcagcagcaccccggcaaggcccccaagctgatgatctacgacgtgtccaaccggccctccggcgtgtccaacagattctccggctccaagtccggcaacaccgcctccctgaccatcagcggactgcaggcagaggacgaggccgactactactgctcctcctacacctcctccagcaccagagtgttcggcaccggcacaaaagtgaccgtgctgggccagcccaaggccaacccaaccgtgacactgttccccccatcctccgaggaactgcaggccaacaaggccaccctggtctgcctgatctcagatttctatccaggcgccgtgaccgtggcctggaaggctgatggctccccagtgaaggccggcgtggaaaccaccaagccctccaagcagtccaacaacaaatacgccgcctcctcctacctgtccctgacccccgagcagtggaagtcccaccggtcctacagctgccaggtcacacacgagggctccaccgtggaaaagaccgtcgcccccaccgagtgctca

SEQ ID NO: 49 - protein, 8 aa, Artificial Sequence
  source: /note=“Description of Artificial Sequence Synthetic peptide”
KPSWFTTL

SEQ ID NO: 50 - protein, 8 aa, Gallus sp.
SIINFEKL